(19) United States
(12) Patent Application Publication
(10) Pub. No.: US 2026/0140120 A1
(43) Pub. Date: May 21, 2026

(54) SPATIALLY-MULTIPLEXED FLUORESCENCE IMAGING

(71) Applicant: Quantum-Si Incorporated, Branford, CT (US)

(72) Inventor: Todd Rearick, Cheshire, CT (US)

(73) Assignee: Quantum-Si Incorporated, Branford, CT (US)

(21) Appl. No.: 19/393,040

(22) Filed: Nov. 18, 2025

Related U.S. Application Data
(60) Provisional application No. 63/722,569, filed on Nov. 19, 2024.

(51) Int. Cl. G01N 33/68; G01N 21/64

(52) U.S. Cl. CPC G01N 33/6818; G01N 21/6452; G01N 21/6486; G01N 2201/125

(57) ABSTRACT

Aspects of the present disclosure provide improved fluorescence imaging techniques suitable for fluorescence-based analysis (e.g., sequencing) of an excited sample. Some aspects relate to directing an imaging aperture of a fluorescence imaging device towards a sample well region and capturing an image of the sample well region. Some aspects relate to selecting a sample well region and capturing an image of the selected sample well region. Some aspects relate to, during at least a portion of performing a controlled cleavage of a terminal amino acid in a first sample well, capturing a fluorescence image of a second sample well. Some aspects relate to setting a duration of imaging by and/or a location of an imaging aperture of a fluorescence imaging device. In some embodiments, aspects of the present disclosure provide for faster, less expensive, and/or greater control over fluorescence imaging and analysis of a sample.

### RELATED APPLICATIONS

This application claims the benefit under 35 U.S.C. § 119(e) of U.S. Provisional Patent Application Serial No. 63/622,569, filed Nov. 19, 2024, under Attorney Docket No.: R0708.70179US00, and entitled, “SPATIALLY-MULTIPLEXED FLUORESCENCE IMAGING,” which is incorporated by reference herein in its entirety.

### BACKGROUND

Instruments that are capable of massively-parallel analyses of biological or chemical samples are typically limited to laboratory settings because of several factors that can include their large size, lack of portability, requirement of a skilled technician to operate the instrument, power need, need for a controlled operating environment, and cost. When a sample is to be analyzed using such equipment, a common paradigm is to extract a sample at a point of care or in the field, send the sample to the lab, and wait for results of the analysis. The wait time for results can range from hours to days.

### SUMMARY

Some aspects of the present disclosure provide a fluorescence imaging system, comprising: a first sample well region comprising a first sample well configured to support a first portion of a sample; a second sample well region comprising a second sample well configured to support a second portion of the sample; and a fluorescence imaging device configured to: (a) direct an imaging aperture of the fluorescence imaging device toward the first sample well region; (b) after (a), capture, using the imaging aperture, a first image of the first sample well region; (c) after (b), redirect the imaging aperture toward the second sample well region; and (d) after (c), capture, using the imaging aperture, a second image of the second sample well region.

Some aspects of the present disclosure provide a method of performing fluorescence imaging of a sample using a fluorescence imaging device, the sample having a first sample portion supported by a first sample well of a first sample well region and a second sample portion supported by a second sample well of a second sample well region, and the method comprising: (a) directing an imaging aperture of the fluorescence imaging device toward the first sample well region; (b) after (a), capturing, using the imaging aperture, a first image of the first sample well region; (c) after (b), redirecting the imaging aperture toward the second sample well region; and (d) after (c), capturing, using the imaging aperture, a second image of the second sample well region.

Some aspects of the present disclosure provide a fluorescence imaging system, comprising: a first sample well region comprising a first sample well configured to support a first portion of a sample; a second sample well region comprising a second sample well configured to support a second portion of the sample; and a fluorescence imaging device configured to: select a sample well region from among the first sample well region and the second sample well region; and capture an image of the sample well region.

Some aspects of the present disclosure provide a method of performing fluorescence imaging of a sample using a fluorescence imaging device, the sample having a first sample portion supported by a first sample well of a first sample well region and a second sample portion supported by a second sample well of a second sample well region, and the method comprising: selecting, by a fluorescence imaging device, a sample well region from among the first sample well region and the second sample well region; and capturing, by the fluorescence imaging device, an image of the sample well region.

Some aspects of the present disclosure provide a method of polypeptide sequencing, the method comprising: (a) capturing, by a fluorescence imaging device, a fluorescence image of a first sample well; and (b) in a second sample well, performing a controlled cleavage of a terminal amino acid of a polypeptide immobilized to a surface of the second sample well, the controlled cleavage comprising catalyzing removal of the terminal amino acid of the polypeptide, wherein (a) is performed during at least a portion of (b).

Some aspects of the present disclosure provide a fluorescence imaging system, comprising: a fluorescence imaging device configured to capture a fluorescence image of a sample well region located within an imaging aperture of the fluorescence imaging device over a duration of imaging; and a control circuit configured to set the duration of imaging and/or a location of the imaging aperture.

Some aspects of the present disclosure provide a method of performing fluorescence imaging of a sample using a fluorescence imaging device, the sample being located in a sample well region during the fluorescence imaging, and the method comprising: setting, by a control circuit, a duration of imaging and/or a location of an imaging aperture of the fluorescence imaging device; and capturing, by the fluorescence imaging device, over the duration of imaging, while the sample well region is located within the imaging aperture of the fluorescence imaging device, a fluorescence image of the sample well region.

Some aspects of the present disclosure provide a fluorescence imaging device, comprising: a first fluorescence camera configured to capture light contained within a first optical band; a second fluorescence camera configured to capture light contained within a second optical band; and optical components configured to: receive fluorescent light emitted by a sample, the fluorescent light comprising a first portion having content contained within the first optical band and a second portion having content contained within the second optical band; provide the first portion of the fluorescent light to the first fluorescence camera; and provide the second portion of the fluorescent light to the second fluorescence camera.

Some aspects of the present disclosure provide a fluorescence imaging system, comprising: a fluorescence imaging device configured to capture fluorescent light emitted from a sample, the fluorescent light comprising a first portion having content contained within a first optical band and a second portion having content contained within a second optical band; and processing circuitry configured to determine fluorescence information of the sample based on a relationship between intensity of the first portion of the fluorescent light and intensity of the second portion of the fluorescent light.

Some aspects of the present disclosure provide a fluorescence imaging device, comprising: a first fluorescence camera; a second fluorescence camera; and optical components configured to receive fluorescent light emitted by a sample and divide the fluorescent light between a first optical path that includes the first fluorescent camera and a second optical path that includes the second fluorescent camera.

Some aspects of the present disclosure provide a fluorescence imaging system comprising: a plurality of sample well regions; a controller configured to: select a sample well region of the plurality of sample well regions; dispense a sample, reagent and/or buffer into the sample well region; and a fluorescence imaging device configured to capture fluorescent light emitted from the sample well region following dispensation of the sample, reagent, and/or buffer.

Some aspects of the present disclosure provide a fluorescence imaging and/or processing system, comprising: processing circuitry configured to: obtain a first image comprising a first plurality of pixel values indicating intensity of fluorescent light received at respective pixels of a fluorescence imaging device; and transform the first image into a second image comprising a second plurality of pixel values indicating intensity of fluorescent light received from respective sample wells of a sample well member.

Some aspects of the present disclosure provide a fluorescence imaging and/or processing system, comprising: processing circuitry configured to: obtain a fluorescence image captured by a fluorescence imaging device indicating fluorescent light emitted from a sample well member; determine, based on the fluorescence image, a position of the fluorescence imaging device with respect to the sample well member; and output a signal indicating an extent of alignment and/or misalignment between the fluorescence imaging device and the sample well member.

The foregoing summary is not intended to be limiting. It should be appreciated that aspects of the disclosure may be implemented individually or in any combination.

### BRIEF DESCRIPTION OF DRAWINGS

FIG. 1 is a block diagram of an example fluorescence imaging system, according to some embodiments.

FIG. 2 is a flow diagram of an example fluorescence imaging method including setting a duration of imaging and/or a location of an imaging aperture, according to some embodiments.

FIG. 3A is a block diagram of an example fluorescence imaging system in which a fluorescence imaging device selects and captures a first image of a first sample well region, according to some embodiments.

FIG. 3B is a block diagram of the example fluorescence imaging system of FIG. 3A in which the fluorescence imaging device selects and captures a second image of a second sample well region, according to some embodiments.

FIG. 3C is a block diagram of the sample well member and fluorescence imaging device of FIGS. 3A-3B in which an imaging aperture of the fluorescence imaging device is smaller than a combined area of sample regions of the sample well member, according to some embodiments.

FIG. 3D is a top view of an example sample well region of the sample well member of FIG. 3C including a plurality of sample wells, according to some embodiments.

FIG. 3E is a top view of an alternative example sample well region that may be included in the sample well member of FIG. 3C, according to some embodiments.

FIG. 3F is a top view of the sample well region of FIG. 3E having a projection of pixels in a fluorescence image superimposed thereon, according to some embodiments.

FIG. 4A is an example fluorescence image that may be generated using a fluorescence imaging device described herein, according to some embodiments.

FIG. 4B is a graph of intensity over time that may be obtained, at least in part, using the fluorescence image of FIG. 4A, according to some embodiments.

FIG. 5 is a side view of a portion of a fluorescence imaging system including a sample well member and a fluorescence imaging device, according to some embodiments.

FIG. 6A is a perspective view of a semiconductor wafer including a plurality of optical components configured to deliver excitation light to sample well regions of a sample well member, according to some embodiments.

FIG. 6B is a perspective view of a semiconductor die of the semiconductor wafer of FIG. 6A on a package substrate selectively propagating excitation light through one of four excitation ports of the semiconductor die, according to some embodiments.

FIG. 7A is a perspective view of a portion of a fluorescence imaging system including a sample well member including a plurality of sample well regions and housing components configured to propagate inserted matter into the sample well regions, respectively, according to some embodiments.

FIG. 7B is a side view of a sample well of a sample well region of the sample well member of FIG. 7A, according to some embodiments.

FIG. 8 is a flow diagram of an example fluorescence imaging method including applying controlled cleavage to and performing fluorescence imaging of a first sample well region and applying controlled cleavage to and performing fluorescence imaging of a second sample well region, according to some embodiments.

FIG. 9 is a flow diagram of an example fluorescence imaging method including capturing a fluorescence image of a first sample well region during at least a portion of performing a controlled cleavage in a second sample well region, according to some embodiments.

FIG. 10 is a block diagram of an example fluorescence imaging system including a fluorescence imaging device configured to direct an imaging aperture towards a sample well region and capture an image of the sample well region using the imaging aperture, according to some embodiments.

FIG. 11 is a flow diagram of an example fluorescence imaging method including selecting a sample well region and capturing an image of the sample well region, according to some embodiments.

FIG. 12 is an optical diagram of an example fluorescence imaging system including a fluorescence imaging device configured to direct an imaging aperture towards a sample well region and capture an image of the sample well region using the imaging aperture, according to some embodiments.

FIG. 13 is an optical flow diagram of an alternative example fluorescence imaging system including a fluorescence imaging device including a plurality of fluorescence cameras, according to some embodiments.

FIG. 14A is a plot of normalized fluorescent light intensity vs. ratio of intensity of a portion of fluorescent light in a first optical band with respect to intensity of a portion of the fluorescent light in a second optical band, according to some embodiments.

FIG. 14B is a plot of fluorescent light intensity vs. ratio of intensity of a portion of fluorescent light in a first optical band with respect to intensity of a portion of the fluorescent light in a second optical band, according to some embodiments.

FIG. 14C is a plot of fluorescent light intensity vs. ratio of intensity of a portion of fluorescent light in a first optical band with respect to intensity of a portion of the fluorescent light in a second optical band a plurality of fluorescently tagged DNA species, according to some embodiments.

FIG. 15A is an optical diagram of an example fluorescence imaging device including a plurality of fluorescence cameras, according to some embodiments.

FIG. 15B is a perspective view of an example fluorescence imaging device implemented based on the optical diagram of FIG. 15A, according to some embodiments.

FIG. 15C is a side view of the fluorescence imaging device imaging device of FIG. 15B, according to some embodiments.

FIG. 16A is a cross-section of integrated devices of a fluorescence imaging system containing a sample well member and a fluorescence imaging device, respectively, according to some embodiments.

FIG. 16B is a cross-section of alternative integrated devices of a fluorescence imaging system containing a sample well member and a fluorescence imaging device, respectively, according to some embodiments.

FIG. 17A is a magnified cross-section of a portion of the integrated device of FIG. 16B, according to some embodiments.

FIG. 17B is a magnified cross-section of a portion of an alternative integrated device that may be included in the system of FIG. 16B, according to some embodiments.

FIG. 18 is a circuit diagram of an example pixel circuit that may be included in the fluorescence imaging device of FIG. 16A, according to some embodiments.

The features and advantages of the present disclosure will become more apparent from the detailed description set forth below when taken in conjunction with the drawings. When describing embodiments in reference to the drawings, directional references (“above,” “below,” “top,” “bottom,” “left,” “right,” “horizontal,” “vertical,” etc.) may be used. Such references are intended merely as an aid to the reader viewing the drawings in a normal orientation. These directional references are not intended to describe a preferred or only orientation of features of an embodied device. A device may be embodied using other orientations.

### DETAILED DESCRIPTION

Aspects of the present disclosure provide improved fluorescence imaging techniques suitable for fluorescence-based analysis (e.g., sequencing) of an excited sample. Some aspects relate to directing an imaging aperture of a fluorescence imaging device towards a sample well region and capturing an image of the sample well region. Some aspects relate to selecting a sample well region and capturing an image of the selected sample well region. Some aspects relate to, during at least a portion of performing a controlled cleavage of a terminal amino acid in a first sample well, capturing a fluorescence image of a second sample well. Some aspects relate to setting a duration of imaging by and/or a location of an imaging aperture of a fluorescence imaging device. In some embodiments, aspects of the present disclosure provide for faster, less expensive, and/or greater control over fluorescence imaging and analysis of a sample.

The inventors have recognized that existing fluorescence imaging and analysis techniques are time consuming, expensive, and do not provide users with sufficient control over the imaging and analysis process. For example, existing fluorescence imaging and analysis systems include a single sample well region containing a sample, and the entire sample is continuously imaged throughout the imaging and analysis process. One drawback of these approaches is that, since the entire sample is imaged, the imaging aperture of the fluorescence imaging device should be large enough to capture an image of the entire sample (e.g., to avoid having substantial portions of the sample outside the imaging aperture). As the size of the sample to be analyzed increases, the size of the imaging aperture increases in turn, and thus the size of the sample to be analyzed in a given measurement may be limited based on the cost and/or complexity of the fluorescence imaging device (and/or number of imaging devices) needed to obtain an appropriately large imaging aperture.

Another drawback of existing approaches is that the fluorescence imaging device is directed at the entire sample continuously over time, even when no fluorescence may be emitted from at least some parts of the sample. In a fluorescence-based sequencing system, for example, a catalyst for removal of a terminal amino acid and a composition including one or more amino acid recognition molecules may be introduced into a single sample well region. For instance, upon removal of the terminal amino acid, the composition may bind with the next terminal amino acid in the chain and, when excited, emit fluorescent light to be captured in a fluorescence image to identify the next terminal amino acid in the sequence. However, removal of the terminal amino acid typically occurs randomly and continuously throughout the sample, and thus the fluorescence imaging device should be directed at the entire sample continuously over time to maximize the amount of captured fluorescent emissions from each terminal amino acid across the entire sample over time.

To overcome the foregoing drawbacks of existing fluorescence imaging approaches, the inventors have developed improved fluorescence imaging techniques that may be suitable for fluorescence-based analysis (e.g., sequencing) of an excited sample.

Some aspects of the present disclosure relate to selecting a sample well region and capturing an image of the selected sample well region. In some embodiments, a fluorescence imaging system may include a fluorescence imaging device configured to select a sample well region from among a first sample well region configured to support a first portion of a sample and a second sample well region configured to support a second portion of the sample, and to capture the image of the sample well region that was selected. The inventors have recognized that selecting and capturing an image of a sample well region among multiple sample well regions may decouple the size of the sample to be analyzed from the cost (e.g., imaging aperture size and/or resolution) of the fluorescence imaging device. For example, the sample may be distributed among multiple sample well regions that may be selectively imaged using the same fluorescence imaging device. In some embodiments, the fluorescence imaging device may be configured to capture the image only of the selected sample well region and not of the sample well region that was not selected (e.g., due to the imaging aperture of the fluorescence imaging device being smaller than a combined area of the sample well regions).

Some aspects of the present disclosure relate to directing an imaging aperture of a fluorescence imaging device towards a sample well region and capturing an image of the sample well region. In some embodiments, a fluorescence imaging system may include a fluorescence imaging device configured to direct an imaging aperture toward a first sample well region including a first sample well configured to support a first portion of a sample, subsequently capture a first image of the first sample well region using the imaging aperture, then subsequently redirect the imaging aperture toward a second sample well region including a second sample well configured to support a second portion of the sample, and subsequently capture a second image of the second sample well region using the imaging aperture. The inventors have recognized that directing and redirecting an imaging aperture of a fluorescence imaging device towards various sample well regions and capturing fluorescence images using the imaging aperture may decouple the size of the sample to be analyzed from the size of the imaging aperture of the fluorescence imaging device (e.g., permitting use of an imaging aperture smaller than an area occupied by the sample). For example, the sample may be distributed among multiple sample well regions toward which the imaging aperture of the fluorescence imaging device many be directed and redirected to capture a sequence of fluorescence images.

Some aspects relate to setting a duration of imaging by and/or a location of an imaging aperture of a fluorescence imaging device. In some embodiments, a fluorescence imaging system may include a control circuit configured to set a duration of imaging and/or a location of an imaging aperture of a fluorescence imaging device. For example, the fluorescence imaging device may be configured to capture a fluorescence image of a sample well region located within the imaging aperture over the duration of imaging. The inventors have recognized that controlling the duration and/or location of imaging using a fluorescence imaging device may provide greater flexibility in conducting imaging and/or analysis of a sample as compared to continuously imaging the entire sample over a set duration.

In some embodiments, the control circuit may be configured to set the duration of imaging and/or the location of the imaging aperture based on instructions received via a user interface device of the system. For example, the user interface device (e.g., mouse and keyboard, touchscreen, etc.) may permit a user to set the duration and/or location of imaging, providing enhanced user control over the imaging and analysis process.

Some aspects of the present disclosure relate to, during at least a portion of performing a controlled cleavage of a terminal amino acid in a first sample well, capturing a fluorescence image of a second sample well. In some embodiments, a method of polypeptide sequencing may include, during at least a portion of performing a controlled cleavage of a terminal amino acid of a polypeptide immobilized to a surface of a first sample well, capturing a fluorescence image of a second sample well. For example, the controlled cleavage may include catalyzing removal of the terminal amino acid of the polypeptide. The inventors have recognized that performing controlled cleavage and capturing a fluorescence image in different sample wells may reduce the time needed to perform imaging and analysis of a sample. For example, since it may take some time after initiating controlled cleavage (e.g., using an enzyme to catalyze terminal amino acid removal) for the cleavage to occur in a first sample well (e.g., after which fluorescence may be emitted for imaging the next terminal amino acid), another sample well (e.g., having already completed the terminal amino acid removal process) may be imaged in the meantime. For instance, controlled cleavage and imaging may be staggered in time for different sample wells (e.g., sample well regions).

In some embodiments, the first sample well may be located in a first sample well region and the second sample well may be located in a second sample well region. For example, the first sample well region and the second sample well region may include separate flow cells configured to contain the respective portions of the sample, such that the sample does not flow between the first sample well region and the second sample well region, though the sample may flow between sample wells within the respective sample well regions.

It should be appreciated that aspects of the present disclosure may be implemented individually or in any combination.

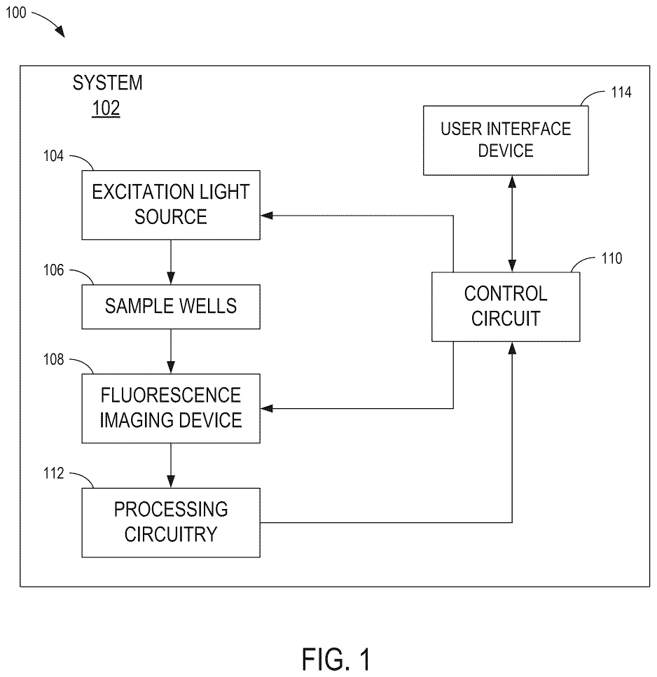

Turning to the figures, FIG. 1 is a block diagram 100 of an example fluorescence imaging system 102, according to some embodiments. As shown in FIG. 1, the fluorescence imaging system includes an excitation light source 104, sample wells 106, and a fluorescence imaging device 108. In some embodiments, excitation light source 104, sample wells 106, and fluorescence imaging device 108 may be included in an instrument (e.g., a sequencing instrument), such as may be used in benchtop applications.

In some embodiments, the sample wells 106 may be configured to support a sample. For example, the sample wells 106 may be sized to contain a small portion of the sample, such as a single molecule, during reactions that produce fluorescent emissions from the sample. In some embodiments, the sample wells 106 may be arranged in multiple sample well regions, which may permit the sample to flow between sample wells 106 within a sample well region, whereas the sample may be inhibited from flowing between a sample well of one sample well region and a sample well of another sample well region.

In some embodiments, a sample well region (e.g., including one or more sample wells 106) may be included within a consumable member (not shown) that is configured to be consumed from supporting a sample. For example, the consumable member may be formed from a material that may deteriorate over time from supporting a sample. For instance, using a consumable member may increase the flexibility of the fluorescence imaging system to perform imaging and/or analysis of samples of various sizes and/or over various numbers of sample regions as compared to having a fixed member including sample well regions. Alternatively or additionally, a consumable member may reduce the cost of the fluorescence imaging system as compared to having a fixed member that may need to survive many uses and/or flex to accommodate various sample sizes. It should be appreciated, however, that a fixed member may be used in other embodiments without departing from the scope of the present aspects.

In some embodiments, the excitation light source 104 may be configured to illuminate a sample well region (e.g., including the sample well 106) to excite a portion of a sample to emit fluorescent light that the fluorescence imaging device 108 may be configured to capture in a fluorescence image. For example, the excitation light source 104 may include a laser configured to produce excitation light at a wavelength and power level suitable to excite fluorescence in a particular sample. According to various embodiments, the excitation light source 104 may be configured to perform gain control, wavelength control (e.g., to excite fluorescence of a particular sample), modulation, filtering, and/or steering of excitation light.

In some embodiments, the fluorescence imaging device may be configured to capture a fluorescence image of the sample using fluorescence emitted by the sample in response to excitation by the excitation light source 104. For example, the fluorescence imaging device 108 may include optoelectronic components configured to capture fluorescent emissions and produce a fluorescence image therefrom (e.g., in resulting electrical signals). For instance, a fluorescence imaging device 108 may include a camera, and/or an integrated circuit including an array of photodetector pixels configured to capture light received from a sample well region to generate a fluorescence image. In some embodiments, a fluorescence imaging device 108 may be configured to convert received fluorescent light into electrical signals to produce a fluorescence image. In some embodiments, the fluorescence imaging may include optical components configured to focus fluorescent emissions towards an image sensor (e.g., photodetector pixel array) and/or filter out undesired light from the fluorescent emissions prior to reaching the image sensor.

In some embodiments, the fluorescence imaging device 108 may be configured to perform arrival time-based and/or wavelength-based discrimination of received fluorescent light, which may be encoded into the produced electrical signals for downstream analysis of the received fluorescent light. For example, wavelength-based discrimination may be achieved by capturing portions of fluorescent light from the sample in respective portions of a fluorescence camera and/or respective fluorescent cameras. In the same or another example, photodetector pixels of a fluorescence imaging device 108 may be configured to discriminate between (e.g., segregating into different electrical signals) received light based upon a wavelength of the light and/or based upon a time at which the light arrives at the array of pixels. For instance, time-based discrimination may be performed by storing photogenerated charge carriers corresponding to received light in time bins depending on the time at which the light was received. Similarly, wavelength-based discrimination may be performed using photodetector pixels of a fluorescence imaging device 108 by storing photogenerated charge carriers corresponding to received light in wavelength bins depending on the wavelength of the received light, and/or by receiving the light at multiple (e.g., narrowband) photodetectors configured to generate charge carriers in response to different wavelengths of light. In some embodiments, the fluorescence imaging device 108 may be alternatively or additionally configured to reject some or all light from the excitation light source 104, which may not contribute towards analysis of the fluorescent emissions from the sample.

In some embodiments, the fluorescence imaging device 108 may alternatively or additionally include readout circuitry configured to produce electrical signals indicating fluorescent emissions captured in fluorescence images by the fluorescence imaging device 108, such as to provide the fluorescence images downstream for analysis. For example, the readout circuitry may be provided for some or all photodetectors (e.g., to provide individual and/or aggregate electrical signals) and/or may be coupled to analog-to-digital conversion (ADC) circuitry to produce the electrical signals as digital signals (e.g., for processing using a digital signal processor such as a general-purpose central processing unit (CPU)).

As shown in FIG. 1, the fluorescence imaging system 102 further includes a control circuit 110. In some embodiments, the control circuit 110 may be configured to set a duration of imaging and/or a location of an imaging aperture of the fluorescence imaging device 108 (e.g., within which a sample well region is located over the duration of imaging). For example, the control circuit 110 may be configured to set the duration of imaging including a plurality of sub-durations of imaging a plurality of sample well regions, respectively. For instance, a first sample well region may be imaged in a first sub-duration (e.g., while controlled cleavage is performed in a second sample well region, as described further herein) and a second sample well region may be imaged in a second sub-duration.

In some embodiments, the plurality of sub-durations of imaging the plurality of sample well regions may include different sub-durations. For example, different types of measurements (e.g., sequencing measurements) result in different sub-durations between imaging respective sample well regions. As one example, where a known protein is to be analyzed by capturing fluorescence images of predetermined portions of an amino acid sequence, a longer imaging duration may be used for the predetermined portions of the amino acid sequence (e.g., targeted for analysis) than for other portions of the amino acid sequence. Alternatively or additionally, where measurements are to be performed by discriminating between recognition molecules by fluorescence wavelength alone (e.g., as compared to alternatively or additionally using fluorescence lifetime and/or pulse duration), imaging may take place over a shorter duration due to relative ease of distinguishing between recognition molecules.

In some embodiments, the control circuit 110 may be configured to set the duration of imaging (and/or sub-durations) based on a selected type of sequencing measurement of a plurality of predetermined types of sequencing measurements. For example, the control circuit 110 may be configured to set the duration of imaging in a range from a first duration based on a first predetermined type of sequencing measurement to a second duration based on a second predetermined type of sequencing measurement. For instance, some types of sequencing measurements may correspond to different durations of imaging than other types of sequencing measurements. According to various embodiments, the plurality of predetermined types of sequencing measurements may be selected from a group consisting of predetermined polypeptide sequencing, sequencing using only wavelength-based recognition molecule discrimination, and sequencing using at least time (e.g., lifetime and/or pulse duration-based recognition molecule discrimination). In some embodiments, the control circuit 110 may be configured to set a location of imaging (e.g., of one or more sample well regions) based on a number of sample well regions being used for a selected type of measurement and/or for respective measurements.

In some embodiments, the control circuit 110 may be configured to set a duration of imaging (and/or sub-durations) at least in part by controlling the timing and/or duration over which the sample is illuminated with excitation light, and/or a time at and/or duration over which the fluorescence imaging device 108 captures a fluorescence image of the sample. For instance, the control circuit 110 may be configured to control the excitation light source to illuminate the sample following controlled cleavage of the sample, as is described further herein, and the control circuit 110 may be configured to control the fluorescence imaging device 108 to capture a fluorescence image of the sample starting at and/or shortly after illumination of the sample. According to various embodiments, the control circuit 110 may be configured to generate and provide a control signal (e.g., a waveform having a controlled timing and/or duration) to the excitation light source 104 and/or to the fluorescence imaging device 108 which may be used (e.g., directly or indirectly) to control timing and/or duration of illumination by the excitation light source 104 and/or image capture by the fluorescence imaging device 108, and/or the control circuit 110 may be configured to provide commands to the excitation light source 104 and/or the fluorescence imaging device 108, which may be configured to set a timing and/or duration of illumination and/or image capture.

In some embodiments, the control circuit 110 may be configured to set a plurality of locations of the imaging aperture of the fluorescence imaging device 108 corresponding to a plurality of sample well regions, respectively. For example, the control circuit 110 may be configured to control the excitation light source 104 to illuminate a selected sample well region and to control the fluorescence imaging device 108 to capture fluorescent emission from the selected sample well region in response to excitation by the illumination. According to various embodiments, the control circuit 110 may be configured to generate and send a control signal (e.g., indicating a selected sample well region) to optically and/or electronically steer the excitation light source 104 and/or the fluorescence imaging device 108 towards the selected sample well region. For instance, where the excitation light source 104 and/or the fluorescence imaging device 108 include motorized components, the control signal may be configured to control the motorized component(s) to move the excitation light source 104 and/or the fluorescence imaging device 108 into position to illuminate and/or capture an image of the selected sample well region.

As shown in FIG. 1, the fluorescence imaging system 102 further includes processing circuitry 112. In some embodiments, the processing circuitry 112 may be configured to determine fluorescence information of a sample in a sample well region using a fluorescence image captured by the fluorescence imaging device 108. For example, the processing circuitry 112 may be configured to determine fluorescence information of the sample based on electrical signals produced by the fluorescence imaging device 108 in response to capturing fluorescent emissions from the sample during a duration of imaging. According to various examples, the processing circuitry 112 may be configured to determine relative intensity of portions of captured fluorescent light in respective optical bands, a fluorescence wavelength (e.g., using wavelength-based discrimination by the fluorescence imaging device), and/or a fluorescence lifetime and/or pulse duration (e.g., using time-based discrimination by the fluorescence imaging device 108) of the sample based on the electrical signals. In some embodiments, the processing circuitry 112 may be configured to perform sequencing of the sample based on multiple fluorescence images produced over time, such as by repeatedly imaging the sample over time and analyzing the sequence of images produced therefrom. In some embodiments, the processing circuitry 112 may be in wired communication with the fluorescence imaging device 108, such as having at least a portion of the processing circuitry 112 on a same circuit board as the fluorescence imaging device 108 and/or coupled via a cable. In other embodiments, the processing circuitry 112 may be in wireless communication with the fluorescence imaging device 108, such as when the processing circuitry 112 is implemented, at least in part, in the cloud.

In some embodiments, the control circuit 110 may be configured to control the excitation light source 104 and/or the fluorescence imaging device 108 based at least in part on analysis communicated from the processor. For example, the processing circuitry 112 may be configured to provide information to the control circuit 110, such as indicating when fluorescent emissions are detected in a fluorescence image. For instance, the processing circuitry 112 and the control circuitry may be located together within an instrument, including for example having the control circuitry 110 and the processing circuitry 112 implemented using at least some overlapping hardware, though in other embodiments the control circuit 110 and processing circuitry 112 may be entirely separate and in (e.g., wired and/or wireless) communication.

As shown in FIG. 1, the fluorescence imaging system 102 further includes a user interface device 114. In some embodiments, the user interface device 114 may be configured to receive input from a user, such as may be used to control the fluorescence imaging system 102 by communicating the input to the control circuit 110. For example, the input may indicate a selection of a type or mode of analysis to be performed by the fluorescence imaging system 102, such as a particular type of sequencing (e.g., wavelength and/or pulse duration-based) to be performed and/or a number and/or identity of recognition molecules to be used for sequencing. For instance, some types or modes of analysis may use different numbers of sample well regions and/or different durations of controlled cleavage and/or imaging, as described further herein. According to various embodiments, the user interface device 114 may include a mouse, keyboard, remote, touchscreen, and/or button suitable for receiving input from a user.

In some embodiments, the control circuit 110 may be configured to set a duration of imaging and/or a location of an imaging aperture of the fluorescence imaging device 108 based on instructions received via the user interface device 114. For example, the instructions may indicate a selected type of measurement to be performed by the fluorescence imaging system 102, and the duration of imaging and/or the location of the imaging aperture may be based on the selected type of measurement. For instance, the user interface device 114 may be configured to receive a selection of a type or mode of analysis (e.g., including and/or corresponding to a type or mode of sequencing), which may cause the user interface device 114 to communicate the selection to the control circuit 110 to carry out setting the duration and/or location.

It should be appreciated that while some embodiments of the system 102 of FIG. 1 may be included within an instrument, other embodiments may be implemented using a distributed architecture. For example, fluorescence image data may be uploaded (e.g., over a network) from the fluorescence imaging device 108 (e.g., implemented within an instrument) to processing circuitry 112 implemented in the cloud for analysis. For instance, the control circuit 110 (e.g., implemented within the instrument) may be configured to receive input from the cloud (e.g., over the network).

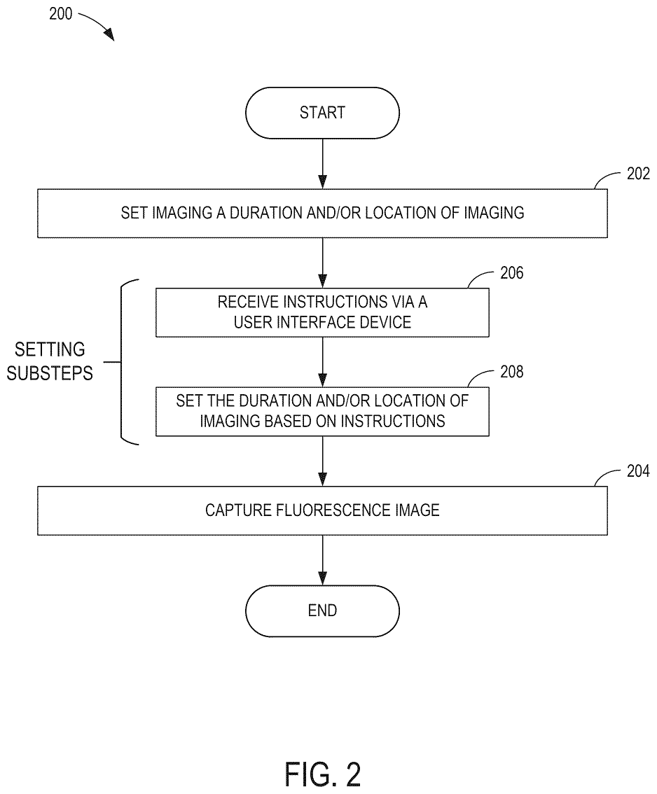

FIG. 2 is a flow diagram of an example fluorescence imaging method 200 including setting a duration of imaging and/or a location of an imaging aperture, according to some embodiments. As shown in FIG. 2, the method 200 includes a step 202 of setting a duration and/or location of imaging and a step 204 of capturing a fluorescence image.

In some embodiments, the method 200 of FIG. 2 may be performed using the fluorescence imaging system 102 of FIG. 1. For example, the step 202 of setting the duration and/or location of imaging may include setting a duration of imaging and/or a location of an imaging aperture of the fluorescence imaging device 108 of the system of FIG. 1, and/or the step 204 of capturing the fluorescence image may be of a sample well region performed over the duration of imaging while the sample well region is located within the imaging aperture of the fluorescence imaging device 108. For instance, the method 200 of FIG. 2 may perform at least a portion of a sequencing measurement of a sample, such as using a sequencing instrument.

In some embodiments, the method 200 of FIG. 2 may further include a step 206 of receiving instructions via a user interface device 114 (e.g., of the system 102 of FIG. 1). The method of FIG. 2 may include a step 208 of setting the duration of imaging and/or the location of the imaging aperture may be based on the instructions. For instance, the instructions may indicate a selected type of measurement to be performed, and the duration of imaging and/or the location of the imaging aperture may be set based on the selected type of measurement, such as described herein including in connection with FIG. 1.

In some embodiments, the control circuit 110 may set a plurality of locations of the imaging aperture corresponding to a plurality of sample well regions, respectively, and/or the control circuit 110 may set the duration of imaging including a plurality of sub-durations of imaging the plurality of sample well regions, respectively. For example, the plurality of sub-durations of imaging the plurality of sample well regions may include different sub-durations, such as described herein including in connection with FIG. 1. For instance, setting the duration of imaging may be based on a selected type of sequencing measurement of a plurality of predetermined types of sequencing measurements, such as in a range from a first duration based on a first predetermined type of sequencing measurement to a second duration based on a second predetermined type of sequencing measurement.

In some embodiments, the method 200 of FIG. 2 may further include determining, by a processing device (e.g., of the system 102 of FIG. 1), fluorescence information of a sample in the sample well region using the fluorescence image, such as described herein including in connection with FIG. 1.

In some embodiments, the method 200 of FIG. 2 may further include illuminating, by an excitation light source 104 (e.g., of the system of FIG. 1), the sample well region to excite a portion of the sample to emit fluorescent light that the fluorescence imaging device 108 captures in the fluorescence image. For example, capturing the fluorescence image may include capturing, by an array of pixels of an integrated circuit of the fluorescence imaging device 108, light received from the sample well region to generate the fluorescence image. For instance, capturing the fluorescence image may include discriminating, by pixels of the array of pixels, between capturing and rejecting received light based upon a wavelength of the light and/or based upon a time at which the light arrives at the array of pixels, such as described herein including in connection with FIG. 1.

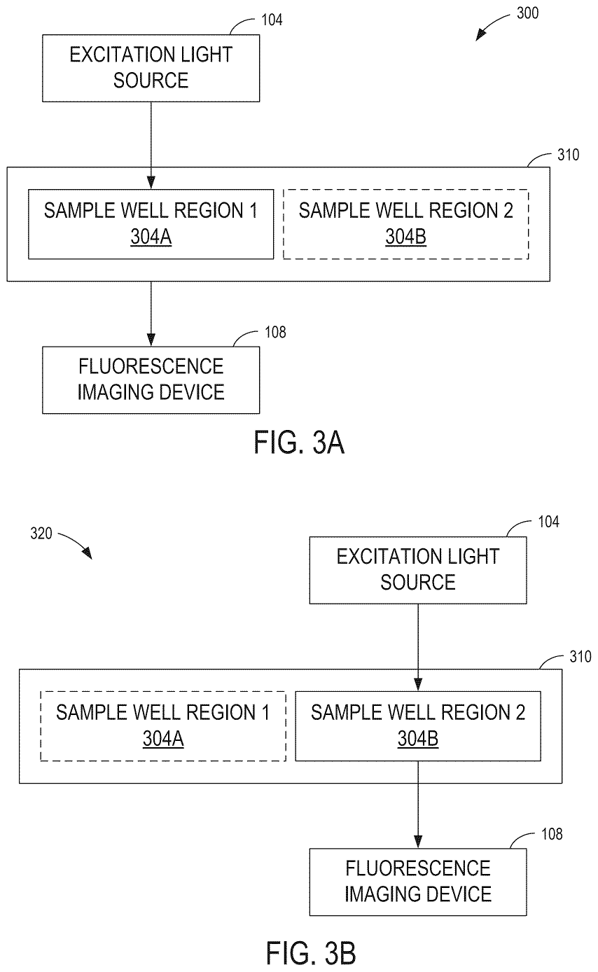

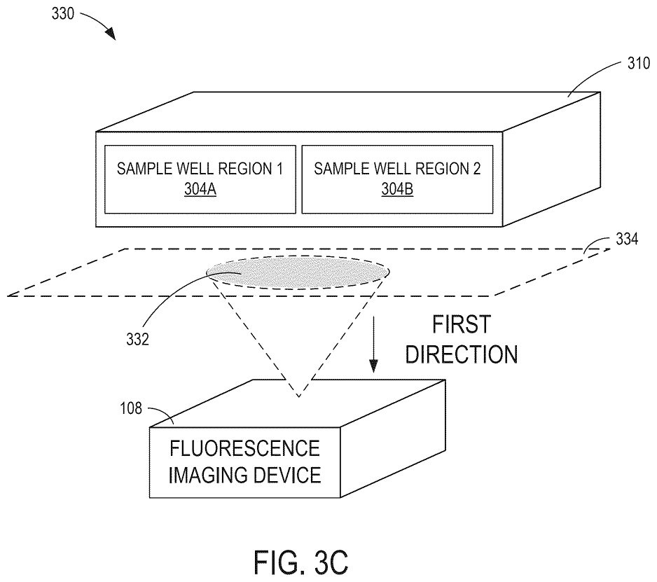

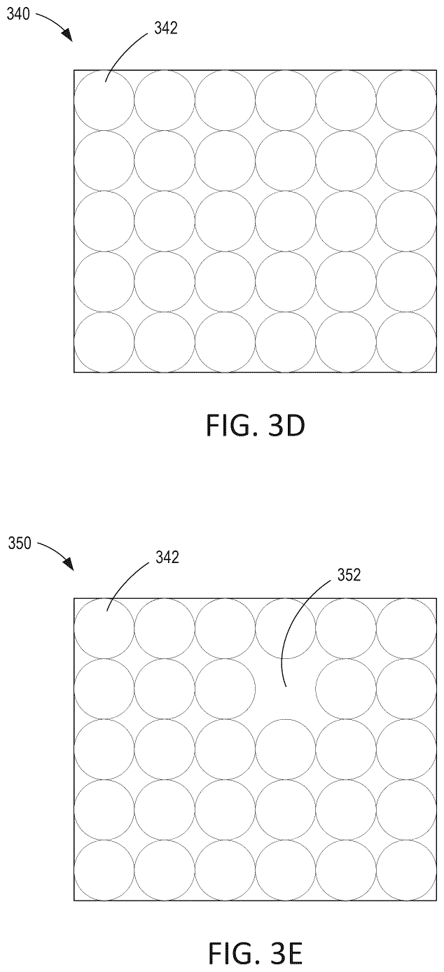

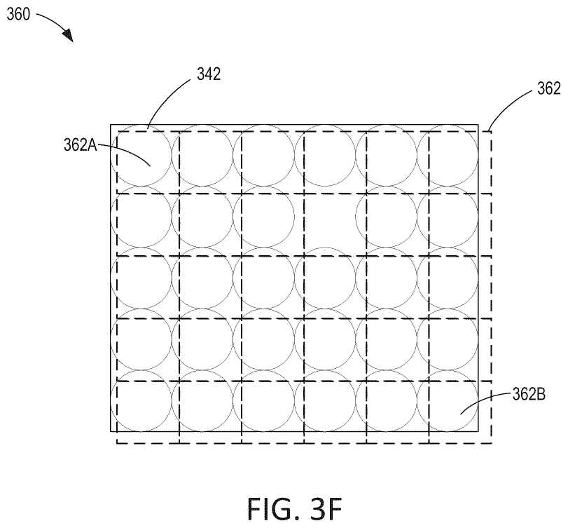

FIG. 3A is a block diagram of an example fluorescence imaging system 300 in which a fluorescence imaging device 108 selects and captures a first image of a first sample well region 304A, according to some embodiments. FIG. 3B is a block diagram of the example fluorescence imaging system 320 of FIG. 3A in which the fluorescence imaging device 108 selects and captures a second image of a second sample well region 304B, according to some embodiments.

In some embodiments, the fluorescence imaging system 302 of FIG. 3A may be configured in the manner described herein for the system 102 of FIG. 1, such as including an excitation light source 104 and a fluorescence imaging device 108, which may be configured as described herein including in connection with FIG. 1.

Further shown in FIG. 3A, the fluorescence imaging system 102 includes a sample well member 310 including a first sample well region 304A and a second sample well region 304B. In some embodiments, each sample well region 304 may include one or more sample wells 106 such as described herein including in connection with FIG. 1. For example, the first sample well region 304A may include a first sample well configured to support a first portion of a sample and the second sample well region 304B may be configured to support a second portion of the sample. For instance, the sample well member 310 may be configured to permit the sample to flow between sample wells within a sample well region, whereas the sample well member 310 may be configured to inhibit the sample from flowing between a sample well of one sample well region 304 and a sample well of another sample well region 304.

In some embodiments, the sample well member 310 may be configured as a consumable member such as described herein including in connection with FIG. 1. It should be appreciated that a sample well member 310 may include any number of sample well regions without departing from the scope of the present aspects.

In some embodiments, the fluorescence imaging device 108 may be configured to select a sample well region from among the first sample well region 304A and the second sample well region 304B and capture an image of the sample well region 304. For example, as shown in FIG. 3A, the fluorescence imaging device selects 302 and captures a first image of the first sample well region 304A, whereas in FIG. 3B, the fluorescence imaging device 108 selects and captures a second image of the second sample well region 304B. For instance, the first image may not include at least a portion of the second sample well region 304B and/or the second image may not include at least a portion of the first sample well region 304A.

In some embodiments, the excitation light source 104 may be configured to illuminate a sample well region 304 selected by the fluorescence imaging device 108 to excite a portion of the sample to emit fluorescent light that the fluorescence imaging device 108 is configured to capture in the image. For example, in some embodiments, the excitation light source 104 and the fluorescence imaging device 108 may be coupled to a same control circuit, such as shown in FIG. 1, such that the excitation light source 104 may be controlled to illuminate a sample well region 304 that the fluorescence imaging device 108 is configured to select to capture an image. For instance, the excitation light source 104 may be moved and/or controlled to direct excitation light towards the selected sample well region 304. Alternatively or additionally, in some embodiments, the excitation light source 104 may be configured to illuminate multiple and/or all sample well regions 304, and the fluorescence imaging device 108 may be configured only to capture fluorescent light from the selected sample well region(s) 304.

In some embodiments, the system of FIGS. 3A-3B may further include a processing device (not shown) configured to determine fluorescence information of the sample using the first image and/or the second image, such as described herein including in connection with FIG. 1.

While the illustrated embodiment shows two sample well regions 304A, 304B in the sample well member 310, it should be appreciated that any number of sample well regions may be included in a sample well member 310. Moreover, while the illustrated embodiment shows a single sample well region being selected and captured in an image, it should be appreciated that any number of sample well regions 304 may be selected and captured in an image (e.g., selecting two of three sample well regions 304) depending, for example, on the size of the imaging aperture of the fluorescence imaging device 108 and/or the number of fluorescence imaging devices in the system.

FIG. 3C is a block diagram 330 of the sample well member 310 and fluorescence imaging device 108 of FIGS. 3A-3B in which an imaging aperture 332 of the fluorescence imaging device 108 is smaller than a combined area of the sample regions 304 of the sample well member 310, according to some embodiments.

In some embodiments, the fluorescence imaging device 108 may be configured to capture the image(s) using an imaging aperture 332 that is smaller than a combined area of the first sample well region 304A and the second sample well region 304B. For example, the imaging aperture 332 may be defined by a field of view of the fluorescence imaging device 108, which in turn may be defined by the size (e.g., in dimensions parallel to the first plane 334) of an image sensor (e.g., a photodetector array) of the fluorescence imaging device 108 and/or of optical components of the fluorescence imaging device 108 configured to direct light towards the image sensor. For instance, the imaging aperture 332 of the fluorescence imaging device 108 may be at least an area of a photodetector array of the fluorescence imaging device 108, though optical components (e.g., an objective lens) may be configured to focus light received over a larger area onto the photodetector array, thereby expanding the imaging aperture 332 with respect to the photodetector array.

In the illustrated embodiment, the fluorescence imaging device 108 may be configured to capture light in a first direction (shown) and the imaging aperture 332 may be in a first plane 334 that is transverse to the first direction. For example, the fluorescence imaging device 108 may include a photodetector pixel array arranged, at least in part, parallel to the first plane 334 so as to project the imaging aperture 332 along the first direction. Alternatively or additionally, the fluorescence imaging device 108 may include optical components configured to direct light received along the first direction towards an image sensor (e.g., photodetector pixel array).

In some embodiments, the combined area of the first sample well region 304A and the second sample well region 304B may be defined by a total area of the first sample well region 304A and the second sample well region 304B in the first plane 334 in which the imaging aperture 332 lies. For example, in the illustrated embodiment of FIG. 3C, the combined area of the first sample well region 304A and the second sample well region 304B may be in a second plane (not shown) parallel to the first plane 334. For instance, the second plane may be defined, at least in part, by a surface of the sample well member 310 that is parallel to (e.g., and oriented towards) the fluorescence imaging device 108. In some embodiments, the imaging aperture 332 may be smaller than an area of the sample well member 310 that includes the first sample well region 304A and the second sample well region 304B.

While FIG. 3C shows planes of the sample well member 310 and fluorescence imaging device 108 being exactly parallel, it should be appreciated that the first plane 334 and the second plane need not be exactly parallel, as the planes may be at least somewhat askew while still capturing useful fluorescence images using the imaging aperture 332.

FIG. 3D is a top view of an example sample well region 340 of the sample well member 310 of FIG. 3C including a plurality of sample wells 342, according to some embodiments.

As shown in FIG. 3D, sample wells 342 may be arranged in a regular array in a sample well region 340. For example, sample wells 342 in the array may correspond (e.g., individually and/or in groups) to pixels in an integrated device of a fluorescence imaging device 108, which may be arranged in a regular array. It should be appreciated, however, that irregular arrays may be used.

FIG. 3E is a top view of an alternative example sample well region 350 that may be included in the sample well member 310 of FIG. 3C, according to some embodiments.

In some embodiments, a fluorescence imaging and/or processing system may include processing circuitry (e.g., FIG. 1) configured to determine an extent of alignment and/or misalignment between a fluorescence imaging device 108 and a sample well member 350 such as shown in FIG. 3E. For example, determining an extent of alignment and/or misalignment may be used to adjust positioning of the fluorescence imaging device 108 and/or sample well member 310 to obtain a target alignment. In some embodiments, determining and adjusting alignment of a fluorescence imaging device 108 and a sample well member 310 may facilitate selecting and capturing an image of a sample well region of the sample well member 310 as described herein.

In some embodiments, processing circuitry may be configured to obtain a fluorescence image captured by a fluorescence imaging device 108 indicating fluorescent light emitted from a sample well member 310 and determine, based on the fluorescence image, a position of the fluorescence imaging device 108 with respect to the sample well member 310. For example, the processing circuitry may be configured to determine the position based on an indicated location in the fluorescence image of an alignment feature of the sample well member 310. For instance, in FIG. 3E, the sample well region 304 is shown including an alignment feature 352 in which a sample well 342 is not present in a location where sample wells 342 are present in other rows in the same column and in other columns in the same row of the array of the sample well region 304. In some embodiments, a fluorescence image may comprise a plurality of pixel values indicating intensity of fluorescent light received at respective pixels of the fluorescence imaging device 108, such that pixel values in the fluorescence image may indicate a location of an alignment feature 352. For example, in FIG. 3E, substantially no fluorescent light may be received at pixels of a fluorescence imaging device 108 that are optically aligned with the location where the sample well is not present. In alternative or additional examples, a sample well 342 may be included but may be offset (e.g., misaligned) with respect to other sample wells in the sample well region 304 (e.g., in the same row or column), and/or a sample well 342 may be smaller and/or larger than other sample wells 342 in the sample well region 304, which may cause less or more fluorescent light to be received from that sample well 342 as compared to other sample wells 342 in the sample well region 304.

In some embodiments, the processing circuitry may be configured to use an indicated location of an alignment feature in a fluorescence image to determine a position of the fluorescence imaging device with respect to the sample well region 304 based on relative positioning of the alignment feature. For example, the alignment feature 352 may be used to determine which sample wells 342 of the sample well member 310 (e.g., within a sample well region 304) correspond to pixel(s) of the fluorescence image. For instance, pixels of the fluorescence image may be mapped relative to the indicated location of the alignment feature 352 to identify in the fluorescence image which sample well(s) 342 emitted the fluorescent light indicated in a given region of the fluorescence image.

In some embodiments, the processing circuitry may be further configured to determine a target position of the fluorescence imaging device 108 with respect to the sample well member 310, at which intensity indicated in the plurality of pixel values may be substantially maximized and/or at which intensity from each of a plurality of sample wells 342 of the sample well member 310 may be indicated in the plurality of pixel values. For example, at a target position, fluorescent light emitted from the sample well member 310 (e.g., in a selected sample well region 304) may be maximally captured by pixels of the fluorescence imaging device 108, though there may be other aspects of selecting a target position in addition to maximizing capture of fluorescent light, such that a target position may be selected that does not maximize such capture. In the same or another example, at a target position, fluorescent light emitted from each sample well of a sample well region 304 (e.g., a selected sample well region 304) may be captured by the fluorescence imaging device 108, though there may be other aspects of selecting a target position in addition to ensuring capture of fluorescent light from each sample well 342, such that a target position may be selected that does not capture fluorescent light from each sample well 342 of a given sample well region 304.

In some embodiments, the processing circuitry may be configured output a signal indicating an extent of alignment and/or misalignment between the fluorescence imaging device 108 and the sample well member 310. For example, the processing circuitry may be configured to output the signal to a controller configured to adjust the position of the fluorescence imaging device 108 with respect to the sample well member 310 in response to the signal indicating misalignment between the fluorescence imaging device 108 and the sample well member 310. For instance, the controller may be configured to control a motor to adjust the position of the fluorescence imaging device 108 to an extent corresponding to the extent of alignment and/or misalignment between the fluorescence imaging device 108 and the sample well member 310. According to various embodiments, the fluorescence imaging and/or processing system may include and/or may be configured to interface with a controller.

According to various embodiments, the fluorescence imaging and/or processing system may include the fluorescence imaging device 108, and processing circuitry may be configured to obtain the fluorescence image by receiving the fluorescence image from the fluorescence imaging device 108 (e.g., in a fluorescence imaging and processing system), whereas in other embodiments the processing circuitry may be configured to interface with the fluorescence imaging device 108 and/or to receive the fluorescence image via an intermediary component (e.g., in a fluorescence processing system).

FIG. 3F is a top view of the sample well region 360 of FIG. 3E having a projection of pixels 362 in a fluorescence image superimposed thereon, according to some embodiments.

In some embodiments, a fluorescence imaging and/or processing system may include processing circuitry 112 (e.g., FIG. 1) configured to transform a first image, including pixel values indicating fluorescent light received at respective pixels of a fluorescence imaging device 108, into a second image, including pixel values indicating fluorescent light received from respective sample wells of a sample well member. For example, such a transformation may be advantageously employed in a system (e.g., in which a sample well region of a sample well member is selectively imaged) without requiring pixels of a fluorescence imaging device to be exactly aligned with respective sample wells 342 of a sample well member 310. For instance, a system may be employed flexibly and without being too constrained by alignment tolerances, though embodiments described herein are not so limited.

In some embodiments, processing circuitry may be configured to obtain a first image comprising a first plurality of pixel values indicating intensity of fluorescent light received at respective pixels of a fluorescence imaging device 108. For example, in FIG. 3F, a plurality of pixels 362 (e.g., a pixel array) is shown superimposed on the sample well region, such that pixel values obtained from the plurality of pixels 362 may indicate fluorescent light received from corresponding portions of the sample well region 360 that are aligned with (e.g., via intermediate optical components) the pixels. For instance, in FIG. 3F, a top-left pixel 362A of the pixel array 362 is aligned with parts of four sample wells, and thus a pixel value for the top-left pixel in a resulting first image may indicate intensity of fluorescent light received from those parts of the four sample wells. In contrast, in FIG. 3F, a bottom-right pixel 362B of the pixel array 362 is aligned with a part of one sample well, and thus a pixel value for the bottom-right pixel 362B in the resulting first image may indicate intensity of fluorescent light received from that part of the one sample well.

In some embodiments, the processing circuitry 112 may be configured to transform the first image into a second image comprising a second plurality of pixel values indicating intensity of fluorescent light received from respective sample wells of a sample well member 310. For example, the processing circuitry 112 may be configured to transform the first image into the second image at least in part by distributing intensity indicated in the first plurality of pixel values among the second plurality of pixel values. For instance, the processing circuitry 112 may be configured to distribute intensity indicated in a first pixel value of the first plurality of pixel values into a second pixel value and a third pixel value of the second plurality of pixel values. In the illustrated example of FIG. 3F, for instance, intensity indicated in the top-left pixel 362A may be distributed into a neighboring pixel directly below the top-left pixel 362A in the left-most pixel column and into a neighboring pixel directly adjacent the top-left pixel 362A in the top-most pixel row, such that the top-left pixel 362A in the transformed image may indicate intensity of fluorescent light received from a respective sample well. It should be appreciated that the number of pixels in the first image and the second image may be different, such as where a different number of sample wells are captured in the first image (e.g., and/or present in the sample well member) from pixels (e.g., resolution) of the fluorescence imaging device 108.

In some embodiments, the processing circuitry 112 may be configured to distribute intensity indicated in the first plurality of pixel values of the first image among the second plurality of pixel values of the second image based on a determined relationship between light received by the respective pixels of the fluorescence imaging device 108 and light emitted by the respective sample wells 342. For example, the determined relationship between light received by the respective pixels and light emitted by the sample wells 342 may indicate an estimated spatial distribution of emitted light among the pixels, such as based on estimated directional spread of the light from the sample wells. In some embodiments, the processing circuitry 112 may be configured to obtain the determined relationship stored in memory, and/or the processing circuitry may be configured to determine the relationship, such as during a calibration run (e.g., using the sample well member 310 and/or sample well region to be imaged).

In some embodiments, the processing circuitry 112 may be alternatively or additionally configured to distribute intensity indicated in the first plurality of pixel values among the second plurality of pixel values based on a determined alignment between the sample well member 310 and the fluorescence imaging device 108. For example, the processing circuitry 112 may be configured to determine alignment between the sample well member and the fluorescence imaging device 108 based on an indicated location in the first image of an alignment feature 352 of the sample well member 350, such as described herein including in connection with FIG. 3E.

In some embodiments, the second plurality of pixel values may indicate intensity of fluorescent light received from respective sample wells of a sample well region of a plurality of sample well regions of the sample well member. For instance, the fluorescence imaging device 108 may be configured to selectively image regions of the sample well member 310, which may result in the fluorescence imaging device 108 directing an imaging aperture 332 thereof to various sample well regions, such as including the sample well region shown 360 in FIG. 3F.

In some embodiments, the processing circuitry 112 may be alternatively or additionally configured to transform images obtained from multiple fluorescence cameras of a fluorescence imaging device 108 into respective resulting images, values of which may be combined for subsequent processing. For example, the processing circuitry 112 may be configured to obtain, in addition to the first image captured by a first fluorescence camera of the fluorescence imaging device 108, a third image comprising a third plurality of pixel values indicating intensity of fluorescent light received at respective pixels of a second fluorescence camera of the fluorescence imaging device 108. In this example, the processing circuitry 112 may be configured to transform the third image into a fourth image comprising a fourth plurality of pixel values indicating intensity of fluorescent light received from the respective sample wells of the sample well member, such as described herein for transforming the first image into the second image. For instance, the transformation of the third image into the fourth image may vary with respect to the transformation from the first image into the second image, such as based on different resolutions (e.g., pixel counts) of the first fluorescence camera and the second fluorescence camera. In some embodiments, the transformations for the respective fluorescence cameras may use, e.g., different indicated locations of an alignment feature such as due to differences in resolution.

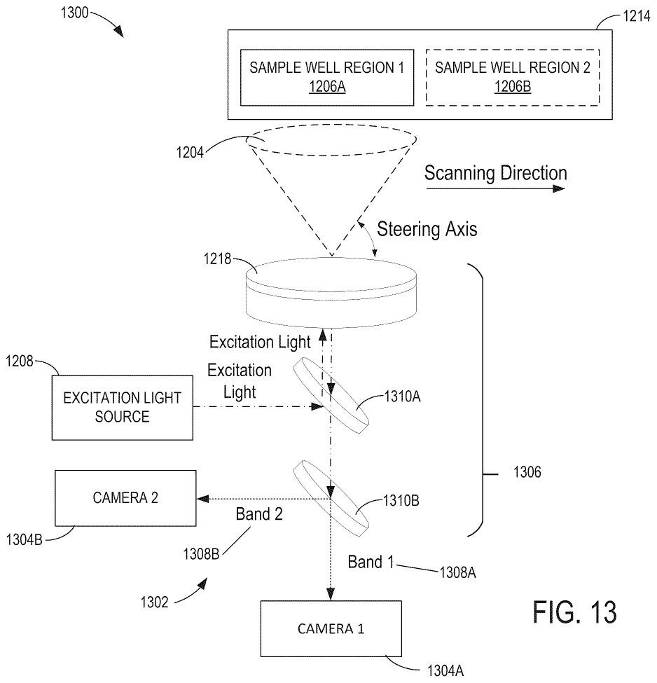

In some embodiments, the first fluorescence camera and the second fluorescence cameras may be configured to capture respective portions of the fluorescent light emitted by the sample wells., such as described further herein including in connection with FIG. 13. For example, the fluorescent light indicated in the first plurality of pixel values may be contained within a first optical band (e.g., in which the first fluorescence camera is configured to capture light) and the fluorescent light indicated in the second plurality of pixel values may be contained within a second optical band (e.g., in which the second fluorescence camera is configured to capture light).

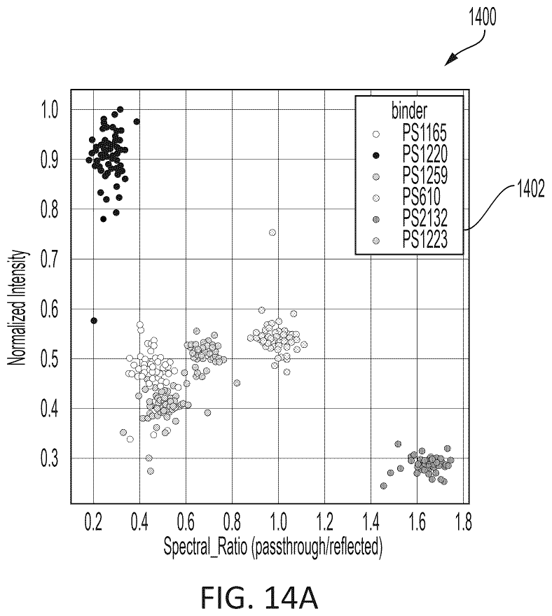

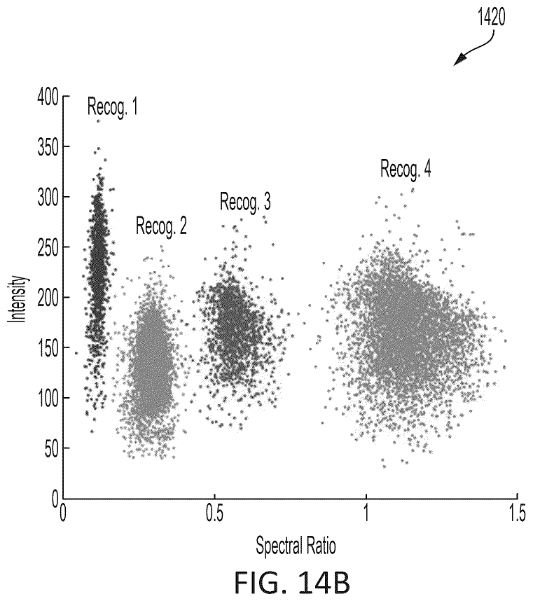

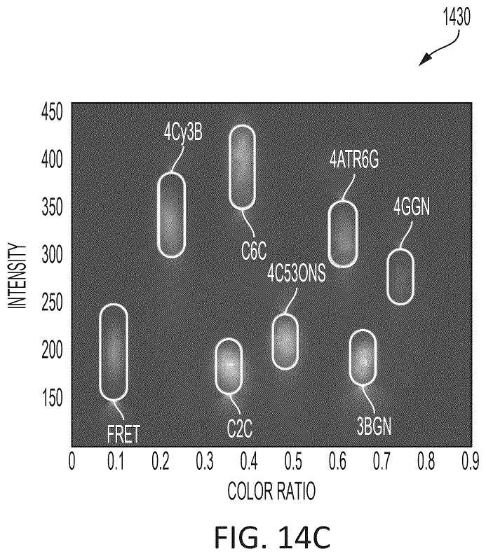

In some embodiments, the processing circuitry 112 may be further configured to combine the second plurality of pixel values of the second image with the fourth plurality of pixel values to determine a total intensity of fluorescent light emitted by each of the respective sample wells and/or a relationship between intensity of the fluorescent light indicated in the second plurality of pixel values and intensity of the fluorescent light indicated in the fourth plurality of pixel values, respectively. For example, the total intensity of fluorescent light emitted by a given sample well as combined across the second image and the fourth image, and/or the relationship between intensity of the fluorescent light emitted by the sample well across the second image and fourth image, may be used to characterize the sample, such as described further herein including in connection with FIGS. 14A-14B.

In some embodiments, the processing circuitry 112 may be configured to combine the second plurality of pixel values with the fourth plurality of pixel values prior to and/or at least in part while receiving, from the fluorescence imaging device 108, a fifth fluorescence image comprising a fifth plurality of pixels indicating intensity of fluorescent light received at the respective pixels of the fluorescence imaging device 108. For example, the processing circuitry 112 may be configured to perform transformation and/or combination of images from multiple fluorescence cameras substantially in real-time, such as opposed to receiving (e.g., at once) a set of images captured over time and performing transformation and/or combination among the set of images after the whole set of images has been received. It should be appreciated, however, that embodiments described herein are not so limited.

According to various embodiments, the fluorescence imaging and/or processing system may further include the sample well member, whereas in other embodiments the processing circuitry 112 may be configured to operate together with a sample well member 310 (e.g., by interfacing with a fluorescence imaging device 108 that is in turn configured to interface with the sample well member).

According to various embodiments, the fluorescence imaging and/or processing system may include the fluorescence imaging device, and processing circuitry may be configured to obtain the first image by receiving the first image from the fluorescence imaging device (e.g., in a fluorescence imaging and processing system), whereas in other embodiments the processing circuitry may be configured to interface with the fluorescence imaging device and/or to receive the first image via an intermediary component (e.g., in a fluorescence processing system).

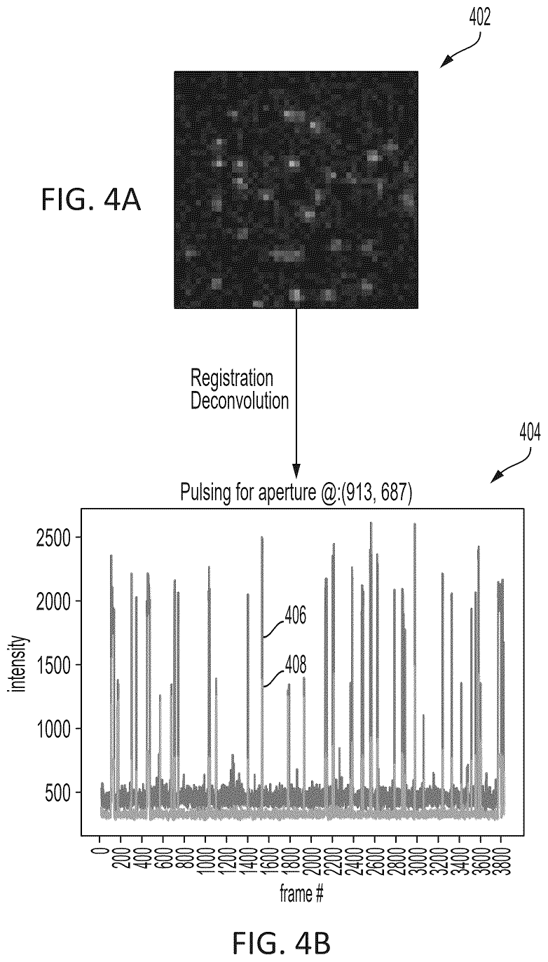

FIG. 4A is an example fluorescence image 402 that may be generated using a fluorescence imaging device 108 described herein, according to some embodiments.

In FIG. 4A, the image 402 includes a plurality of pixels having values indicating intensity of fluorescent light received at respective pixels of a fluorescence camera. For example, the illustrated image may be generated using a fluorescence camera as described further herein.

FIG. 4B is a graph 404 of intensity over time that may be obtained, at least in part, using the fluorescence image 402 of FIG. 4A, according to some embodiments.

As shown in FIG. 4B, the graph of intensity over time corresponds to a pixel (e.g., at x-position 913 and y-position 687) in the image 402 of FIG. 4B) and includes two traces (e.g., a first trace 406 and a second trace 408) having different intensity values in each frame (e.g., captured over time). For example, each trace 406, 408 in the graph 404 of FIG. 4B may be generated by obtaining a first image from a fluorescence camera (e.g., such as shown in FIG. 4A) and transforming the first image into a second image in which pixel values indicate intensity of fluorescent light emitted from a respective sample well (e.g., of a selected sample well region). For instance, the traces 406, 408 may be generated by obtaining images from respective fluorescence cameras (e.g., as described herein including in connection with FIG. 13) and transforming those images into respective resulting images.

As shown in FIG. 4B, the first and second traces 406, 408 have generally overlapping intensity peaks in time but different intensities at those peaks. For example, as described further herein including in connection with FIG. 13, the fluorescence cameras may be configured to receive light contained in respective optical bands, which may cause a pulse of received fluorescent light to cause peaks in both traces, while the division of the fluorescent light into optical bands may result in different intensities in the respective traces.

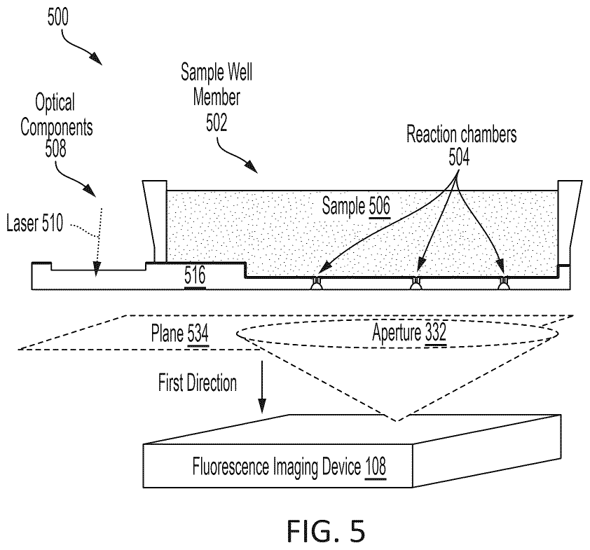

FIG. 5 is a side view of a portion of a fluorescence imaging system 500 including a sample well member 502 and a fluorescence imaging device 108, according to some embodiments.

In some embodiments, the fluorescence imaging system 500 of FIG. 5 may be configured as described herein in connection with FIGS. 3A-3E. For example, in FIG. 5, the fluorescence imaging system 500 includes a sample well member 502 and a fluorescence imaging device 108. In the illustrated embodiment, an imaging aperture 332 of the fluorescence imaging device 108 is directed toward a region of the sample well member 502 that includes three sample wells 504 (“reaction chambers”), and the region of the sample well member includes a liquid sample 506.

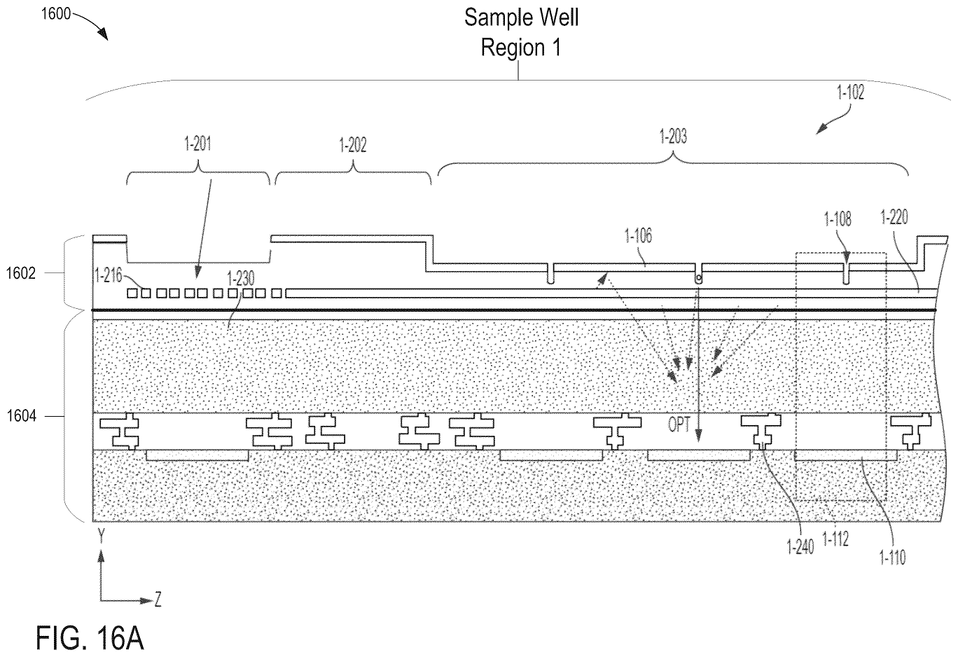

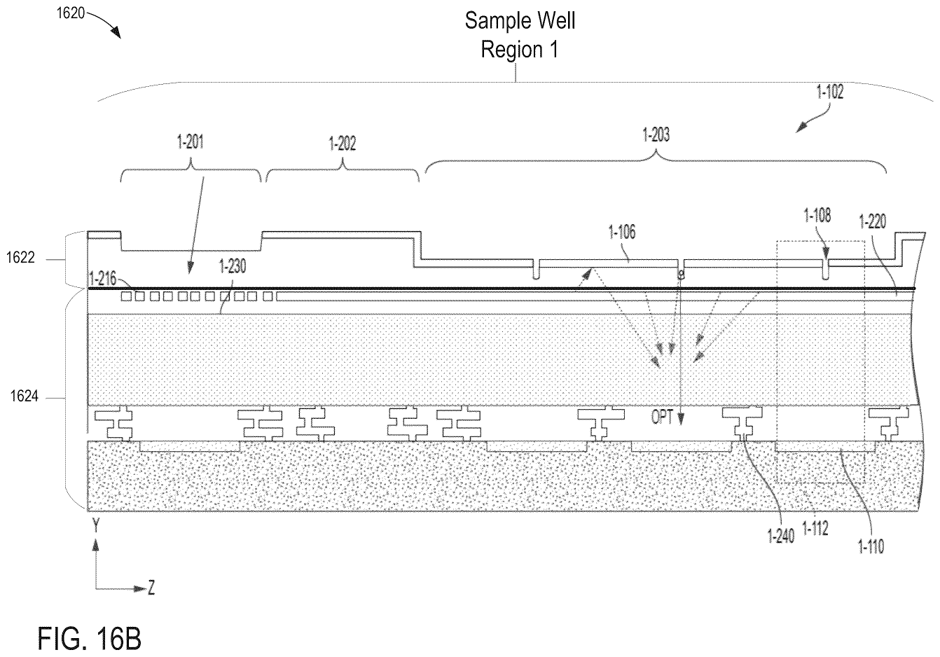

In some embodiments, a sample well member 502 may include and/or may be configured to interface with optical components 508 configured to deliver excitation light 510 into a region 512 of the sample well member 502 to illuminate sample wells within the region. For example, in FIG. 5, the sample well member 502 is shown interfacing with optical components 508, which may be disposed on an integrated device 514 onto which the sample well member 502 may be mounted, and the optical components 508 include a waveguide layer 516 that passes underneath the sample wells 504. For instance, the waveguide layer 516 may terminate at a grating coupler (e.g., FIGS. 16A-16B) configured to receive and propagate excitation light 510 (e.g., from an excitation light source 104) to the sample wells 504 to illuminate the sample 506. In some embodiments, the waveguide layer 516 may be configured to evanescently couple excitation light 510 to the sample wells 504, such as described further herein including in connection with FIGS. 16A-16B. While the optical components 508 are shown in FIG. 5 receiving excitation light 510 (“LASER”) at a side facing the sample well member 502, it should be appreciated that the optical components 508 may be alternatively or additionally configured to receive excitation light 510 at an opposite and/or transverse side from the sample well member 502.

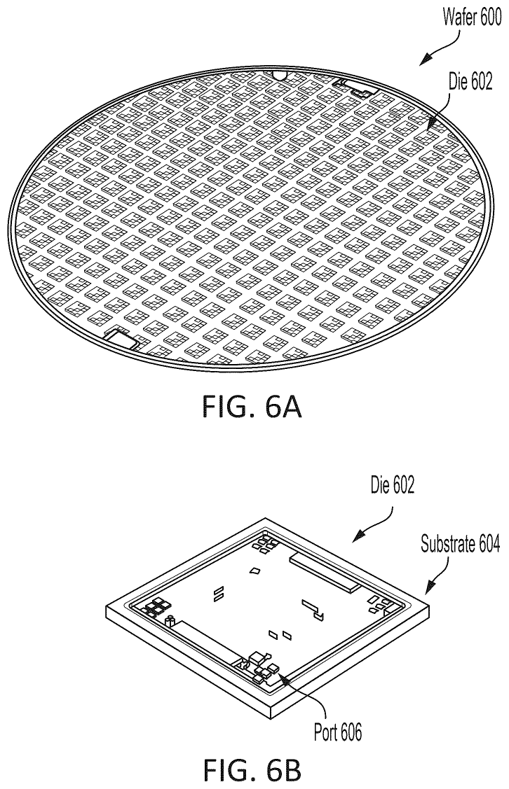

FIG. 6A is a perspective view of a semiconductor wafer 600 including a plurality of optical components configured to deliver excitation light 510 to sample well regions of a sample well member 502, according to some embodiments.

In some embodiments, the semiconductor wafer shown in FIG. 6A may include a plurality of optical component sets configured to be diced into optical component dies 602. For example, each optical component die may include a portion such as shown in FIG. 5 for the optical components 508. For instance, the optical components of the die 602 may include a grating coupler and a waveguide layer 516 configured to receive and propagate excitation light 510 to illuminate a region of a sample well 504 (e.g., mounted over the die 602). In some embodiments, the wafer 600 may be configured to be diced such that multiple sets of optical components 508 are in a single die 602, such as including two or more (e.g., four) sets of optical components 508 in a die 602. For example, a die 602 may include multiple sets of optical components 508 configured to illuminate respective regions of a sample well member 502 supported above the die 602.

FIG. 6B is a perspective view of a semiconductor die 602 of the semiconductor wafer 600 of FIG. 6A on a package substrate 604 selectively propagating excitation light 510 through one of four excitation ports 606 of the semiconductor die 602, according to some embodiments.

In some embodiments, the illustrated die 602 may be configured to illuminate respective regions of a sample well member 502 mounted above the die 602. For example, in FIG. 6B, the die has four quadrants that may be configured to illuminate four respective regions of a sample well member 502. For instance, the four regions of the sample well regions may be selectively illuminated for excitation and capture of fluorescent light using a fluorescence imaging device 108 as described herein. While the illustrated example has four regions, a die 602 may include any number of sets of optical components 508 configured to illuminate any number of respective regions of a sample well member 502, including a single set of optical components 508 configured to illuminate a single region.

As shown in FIG. 6B, the die 602 has four ports 606, of which one is labeled and illuminated with light. For example, the port 606 may include a grating coupler in optical communication with a waveguide layer of the die 602 to propagate light for illuminating a respective sample well region (e.g., overlying the quadrant of the die including the labeled port). In some embodiments, the die 602 may be configured to passively select a port for illumination by receiving light at the selected port 606 (e.g., based on control of the excitation light source 104). Alternatively or additionally, the die 602 may be configured to receive control signals (e.g., propagated via a board on which the packaged die is mounted) that cause the die 602 to select a port 606 for illumination.

In some embodiments, a die 602 that includes optical components 508 configured to illuminate sample well regions of a sample well member 502 may be advantageously implemented to interface with a sample well member 502, allowing the sample well member 502 to be made with low complexity and at low cost, which may facilitate implementing the sample well member 502 as a consumable. It should be appreciated, however, that dies 602 described herein may alternatively or additionally be implemented including sample well regions thereon, and/or may be configured to interface with non-consumable sample well members, as embodiments described herein are not so limited.

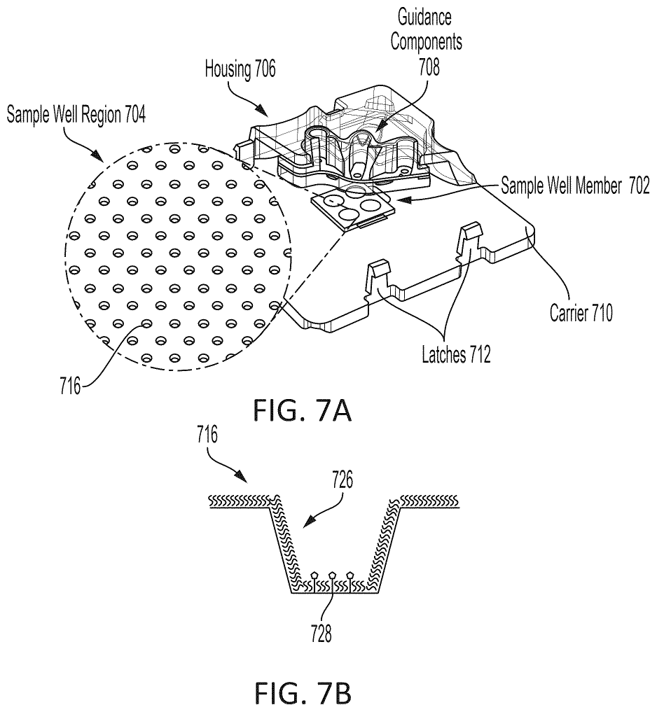

FIG. 7A is a perspective view of a portion of a fluorescence imaging system 700 including a sample well member 702 including a plurality of sample well regions 704 and housing components 706 configured to propagate inserted matter into the sample well regions 704, respectively, according to some embodiments.

In some embodiments, the fluorescence imaging system 700 shown in FIG. 7A may be configured as described herein including in connection with FIGS. 3A-3E and 6A-6B. For example, as shown in FIG. 7A, the system 700 includes a sample well member 702 including multiple sample well regions 704, such as described 700 herein including in connection with FIGS. 3A-3E. As further shown in FIG. 7A, the system 700 includes a housing 706 supporting the sample well member 702 and including guidance components 708. As shown in FIG. 7A, the housing may be mounted onto a carrier 710, wherein the carrier 710 includes a plurality of latches 712 extending towards the housing 706.

In some embodiments, the system 700 of FIG. 7A may be configured for selective dispensation of sample, reagent(s), and/or buffer into regions of the sample well member 702. For example, the system 700 may include a controller (e.g., the control circuit 110 of FIG. 1) configured to select a sample well region 704 and dispense a sample, reagent and/or buffer into the sample well region 704. For example, the controller may be configured to select a first sample well region 704A and dispense the sample, reagent, and/or buffer into the first sample well region 704A and further to select a second sample well region 704B and dispense a sample, reagent, and/or buffer into the second sample well region 704B following dispensation of the sample, reagent, and/or buffer into the first sample well region 704A. For instance, the system may include a motor and/or other mechanism for pouring and/or injecting sample, reagent, and/or buffer into a sample well region 704.

In some embodiments, a controller of the system 700 may be configured to dispense a reagent into the sample well region 704, the reagent comprising at least one sample well member 702 selected from a group consisting of: a composition comprising a terminal amino acid recognition molecule, a composition comprising a catalyst for removal of a terminal amino acid of a polypeptide immobilized to a surface of a sample well in the sample well region 704, and a composition comprising a terminal amino acid modifier. For example, the controller may be configured to dispense the composition comprising the terminal amino acid modifier into the sample well region 704 when the sample well region 704 contains a catalyst for removal of a terminal amino acid of a polypeptide immobilized to a surface of a sample well in the sample well region, and the catalyst may comprise an enzyme having increased catalytic activity for removal of a terminal amino acid having been modified by the terminal amino acid modifier. For instance, the controller may be configured to implement controlled cleavage techniques, as described herein, at least in part by dispensing the reagent(s) into the sample well region.

In some embodiments, a fluorescence imaging device 108 of the system 700 (not shown) may be configured to capture fluorescent light emitted from the sample well region 704 following dispensation of the sample, reagent, and/or buffer. For example, the controller may be configured to dispense a composition comprising a terminal amino acid recognition molecule into the sample well region 704, and the fluorescence imaging device 108 may be configured to capture fluorescent light emitted from the sample well region 704 after the composition comprising the terminal amino acid recognition molecule is dispensed into the sample well region. For instance, the terminal amino acid recognition molecules may be distinguishable in processing fluorescent images of the sample. In some embodiments, the dispensed composition may comprise a plurality of terminal amino acid recognition molecules configured to, when excited, emit different fluorescent light, respectively. For example, the different fluorescent light may comprise fluorescent light having different spectral content, such as may be distinguished using wavelength-based discrimination techniques described herein.

In some embodiments, a controller of the system 700 may be configured to dispense the sample, reagent, and/or buffer into a second sample well region 704 during at least a portion of the fluorescence imaging device 108 capturing fluorescent light emitted from a first sample well region 704A. For example, the controller may be configured to dispense a terminal amino acid recognition molecule and/or a catalyst for removal of a terminal amino acid of a polypeptide immobilized to a surface of a sample well.

In some embodiments, a controller of the system 700 may be configured to maintain a predetermined amount of sample, reagent, and/or buffer in a sample well region 704. For example, the controller may be further configured to detect an amount of the sample, reagent, and/or buffer in the sample well region 704 and, in response to determining that the amount is below a predetermined amount, dispense an additional amount of the sample, reagent, and/or buffer, respectively. For instance, the predetermined amount may be based on a volume of the sample, reagent, and/or buffer for a given experiment and/or the volume of the sample well region.

For example, as shown in FIG. 7A, the guidance components 708 may be configured to guide dispensed sample, reagent(s), and/or buffer to respective regions of the sample well member 702. For instance, in FIG. 7A, the guidance components 708 include holes 714 that are in fluid communication with respective regions of the sample well member 702, which may be configured to dispense sample, reagent(s), and/or buffer into the respective regions. In the illustrated embodiment, the sample well region 704 includes a plurality of sample wells 716 which may contain small portions of the sample, reagent(s), and/or buffer.

In some embodiments, the carrier 710 includes a plurality of latches 712 configured to secure the housing 706. At least one latch 712 may extend from each of an edge of the carrier 710. A plurality of latches 712 may extend from at least one of the edges. The latches 712 may extend from some edges of the carrier 710 but not others, wherein some edges of the carrier 710 have no extending latches 712. In some embodiments, the latches 712 may extend from a surface of the carrier at a distance di from the edge of the carrier in the direction towards a center of the carrier. In such an embodiment, a recess 718 in the carrier recedes towards bases of the latches. In other embodiments, the latches 712 may be offset the edge of the carrier at the distance di from the edge of the carrier without a recess 718.

A latch may extend from the surface of the carrier 710 at a first end 720 of the latch 712. The latch 712 may include a protrusion 722 proximate a second end 724 of the latch 712, wherein the second end 724 is opposite the first end 720. The second end 724 of the latch 712 may include a bevel or a curved surface. The protrusion 722 of the latch 712 may face towards the center of the carrier 710. The protrusion 722 of the latch 712 may face away from the center of the carrier 710. The latches 712 may secure the housing 706 to the carrier 710 using spring forces.

For example, as shown in FIG. 7A, the carrier 710 includes a plurality of latches 712, the plurality of latches 712 including two latches on a first edge of the carrier and two latches on a second edge of the carrier 710, wherein the first edge is opposite the second edge. A third edge and a fourth edge, wherein the fourth edge is opposite the first edge, do not include latches. The latches 712 secure the housing 706 to the carrier 710 by slidably inserting into openings in the housing 706 and engaging with ledges proximate the openings (not shown). The recesses 718 in the carrier at the first end 720 of the latches 712 may guide the fluorescence imaging system 700 to mount on a mechanical stage 1012 (e.g., platform) of the mechanical scanner.

FIG. 7B is a side view of a sample well 716 of a sample well region 704 of the sample well member 702 of FIG. 7A, according to some embodiments. As shown in FIG. 7B, a sample well 716 of a sample well region 704 may be configured to support a sample, reagent(s), and/or buffer. For example, a surface of the sample well region 704 may include surface passivation 726 and may be functionalized 728, such as configured to immobilize a portion (e.g., polypeptide) of a sample in the sample well region.

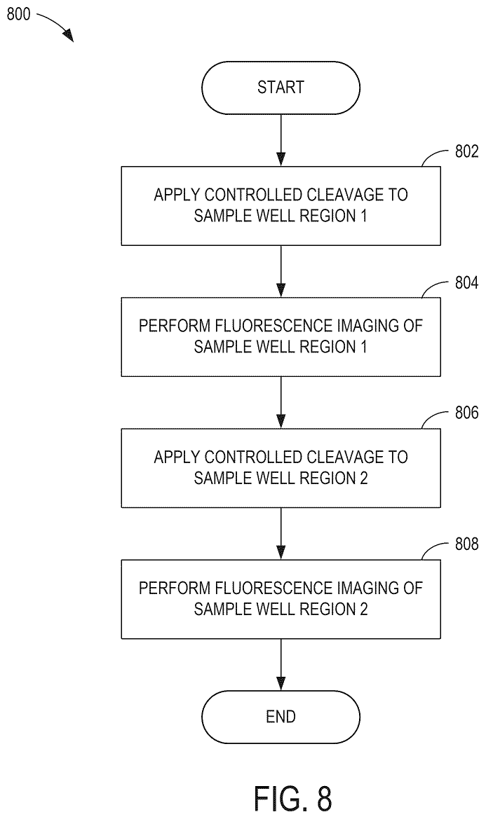

FIG. 8 is a flow diagram of an example fluorescence imaging method 800 including applying controlled cleavage to and performing fluorescence imaging of a first sample well region 304A and applying controlled cleavage to and performing fluorescence imaging of a second sample well region 304B, according to some embodiments.

As shown in FIG. 8, the method includes a step 802 of applying controlled cleavage to a first sample well region 304A, a step 804 of performing fluorescence imaging of the first sample well region 304A, a step 806 of applying controlled cleavage to a second sample well region 304B, and a step 808 of performing fluorescence imaging of the second sample well region 304B. In some embodiments, the method of FIG. 8 may be performed, at least in part, using the system of FIG. 1 and/or FIGS. 3A-3C. For example, the steps of performing fluorescence imaging may be performed using the fluorescence imaging device 108 of the system 102.

In some embodiments, the step 802 of applying controlled cleavage to the first sample well region 304A may include catalyzing removal of a terminal amino acid of a polypeptide immobilized to a surface of a first sample well of the first sample well region 304A. For example, applying controlled cleavage by catalyzing removal of a terminal amino acid of the polypeptide may condition the next terminal amino acid of the polypeptide for binding to one or more terminal amino acid recognition molecules, which in turn may cause the polypeptide to emit fluorescent light indicating the next terminal amino acid when illuminated by excitation light. In contrast, prior fluorescence imaging techniques may include substantially uncontrolled cleavage in which the removal of a terminal amino acid may occur substantially stochastically, making it difficult or impossible to predict when the next terminal amino acid will bind with an amino acid recognition molecule and emit fluorescent light that should be captured in a fluorescence image.

In some embodiments, the step 802 of applying controlled cleavage to the first sample well region 304A may further include modifying the terminal amino acid of the polypeptide prior to catalyzing the removal of the terminal amino acid. For example, modifying the terminal amino acid of the polypeptide may controllably prepare the sample for catalyzing removal of the terminal amino acid. For instance, catalyzing the removal of the terminal amino acid may use an enzyme having increased catalytic activity for removal of the terminal amino acid following modification with respect to before the modification. In some embodiments, modifying the terminal amino acid of the polypeptide as described herein may make removal of the terminal amino acid more precisely predictable in time than when removal occurs substantially stochastically.

In some embodiments, the step 804 of performing fluorescence imaging of the first sample well region 304A may include illuminating the first sample well region 304A with excitation light and capturing fluorescent light emitted by the first sample well region 304A (e.g., using the fluorescence imaging device 108). For example, the step 804 of performing fluorescence imaging of the fist sample well region 304A may be performed after the step of applying controlled cleavage to the first sample well, such as after catalyzing removal of a terminal amino acid of a polypeptide immobilized to a surface of a first sample well of the first sample well region 304A. For instance, the step 804 of performing fluorescence imaging of the first sample well region 304A may further include contacting the polypeptide immobilized to the surface of the first sample well with a composition including one or more terminal amino acid recognition molecules, such as to cause the sample to emit fluorescent light when illuminated by the excitation light. In some embodiments, contacting the polypeptide with such a composition may be performed after controlled cleavage is expected to have occurred, in contrast to prior fluorescence imaging techniques in which cleavage may occur substantially at any time, and thus one or more terminal amino acid molecules should be in place to contact the next terminal amino acid at all times.

In some embodiments, the first sample well region 304A may be selected (e.g., by a fluorescence imaging device 108) for fluorescence imaging based on the step 802 of applying controlled cleavage to the first sample well region 304A. For example, based on the step 802 of applying controlled cleavage to the first sample well region 304A, the first sample well region 304A may be expected to emit fluorescent light when illuminated, which the fluorescence imaging device 108 may be configured to capture in a fluorescence image.

In some embodiments, the steps 806, 808 of applying controlled cleavage to the second sample well region 304B and performing fluorescence imaging of the second sample well region 304B may be performed similarly to the steps of applying controlled cleavage to the first sample well region 304A and performing fluorescence imaging of the first sample well region 304A. For example, the second sample well region 304B may be selected for fluorescence imaging based on applying controlled cleavage to the second sample well region 304B, such as based on an expectation that removal of a terminal amino acid of a polypeptide in the second sample well region 304B has occurred and the next terminal amino acid in the chain will be indicated in emitted fluorescent light following the controlled cleavage (e.g., after contacting the polypeptide with a composition including one or more terminal amino acid recognition molecules).

In some embodiments, steps of the method 800 of FIG. 8 may be repeated, such as to perform sequencing of a sample in the first sample well region 304A and/or in the second sample well region 304B. For example, controlled cleavage may be performed in a sample well region 304 followed by fluorescence imaging of the sample well region 304, after which controlled cleavage may be performed again in the sample well region 304 and subsequent fluorescence imaging of the sample well region may be performed again. For instance, each step of controlled cleavage may remove a terminal amino acid in the chain of a polypeptide such that each step of fluorescence imaging captures the next terminal amino acid in the chain, thereby producing a sequence over repeated steps of controlled cleavage and fluorescence imaging.

In some embodiments, performing controlled cleavage and fluorescence imaging in separate sample well regions 304 may permit different types of analysis (e.g., sequencing) to be performed in the respective sample well regions 304, even where the same sample is present in each sample well region 304. For example, sample well regions 304 may receive different amino acid recognition molecule compositions (e.g., different combinations of amino acid recognition molecules), such as to determine an abundance of a different polypeptide (e.g., protein) in each sample well region. Alternatively or additionally, as described further below, fluorescence imaging of a sample well region 304 may occur during at least a portion of performing controlled cleavage in another sample well region 304.

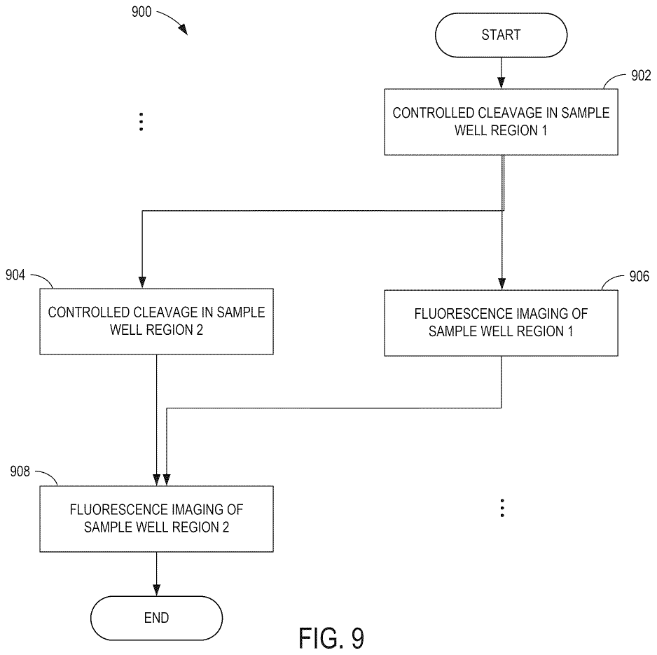

FIG. 9 is a flow diagram of an example fluorescence imaging method including capturing a fluorescence image of a first sample well region during at least a portion of performing a controlled cleavage in a second sample well region, according to some embodiments.

As shown in FIG. 9, the method includes a step of performing controlled cleavage in a first sample well region (e.g., in a first sample well thereof), a step of performing controlled cleavage in a second sample well region (e.g., in a second sample well thereof), and during at least a portion of performing controlled cleavage in the second sample well region, performing fluorescence imaging of the first sample well region. The illustrated method further includes a step of performing fluorescence imaging of the second sample well region.

In some embodiments, the method 900 of FIG. 9 may be performed, at least in part, using the system of FIG. 1 and/or FIGS. 3A-3C. For example, the steps of performing fluorescence imaging may be performed using the fluorescence imaging device 108 of the system 102.

In some embodiments, the steps 902, 904 of performing controlled cleavage in the first sample well region 304A and in the second sample well region 304B and the steps 906, 908 of performing fluorescence imaging of the first sample well region 304A and of the second sample well region 304B may be performed as described herein including in connection with FIG. 8. For example, the step of controlled cleavage in the first sample well region 304A may include performing a controlled cleavage of a terminal amino acid of a polypeptide immobilized to a surface of a first sample well of the first sample well region 304A, including catalyzing removal of the terminal amino acid of the polypeptide. Similarly, the step of performing fluorescence imaging of the first sample well region 304A may be after the controlled cleavage (e.g., and contacting the polypeptide with a composition including one or more terminal amino acid recognition molecules) is expected to have occurred.

In some embodiments, the step of performing fluorescence imaging of the first sample well region 304A may occur during at least a portion of the step of performing controlled cleavage in the second sample well region 304B. For example, at least some fluorescent light from the first portion of the sample in the first sample well region 304A may be received and captured by the fluorescence imaging device 108 (and/or the first sample well region 304A may be at least partially illuminated by the excitation light source 104) during at least a portion of catalyzing removal of a terminal amino acid in the second sample well region 304B, and/or during at least a portion of modifying the terminal amino acid prior to catalyzing removal of the terminal amino acid. For instance, for at least some time used to perform controlled cleavage in the second sample well region 304B (e.g., to condition the next terminal amino acid for fluorescence imaging), the fluorescence imaging device 108 may capture a fluorescence image of the first sample well region 304A (e.g., in which controlled cleavage was previously performed), thereby making use of time in which the second sample well region 304B may not be ready for fluorescence imaging.

As shown in FIG. 9, the illustrated method 900 further includes the step 908 of performing fluorescence imaging of the second sample well region 304B following the step 904 of performing controlled cleavage in the second sample well region. For example, while not shown in FIG. 9, the method 900 may alternate between the steps 902, 904 of performing controlled cleavage in the first sample well region 304A and in the second sample well region 304B and the steps 906, 908 of performing fluorescence imaging of the first sample well region 304A or the second sample well region 304B, depending on which is not currently undergoing controlled cleavage. It should be appreciated that, where more than two sample well regions 304 are included, staggering of controlled cleavage and fluorescence imaging may be performed such that at least one sample well region not undergoing controlled cleavage may be imaged while at least one other sample well region undergoes controlled cleavage.

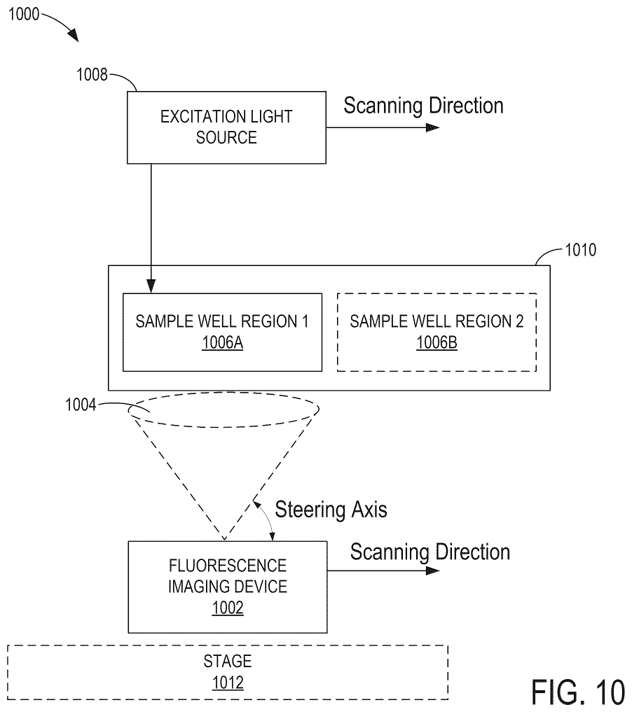

FIG. 10 is a block diagram of an example fluorescence imaging system 1000 including a fluorescence imaging device 1002 configured to direct an imaging aperture 1004 towards a sample well region 1006 and capture an image of the sample well region 1006 using the imaging aperture 1004, according to some embodiments.

In some embodiments, the fluorescence imaging system 1000 shown in FIG. 10 may be configured as described herein including in connection with FIGS. 1 and 3A-3C. For example, as shown in FIG. 10, the illustrated fluorescence imaging system 1000 includes an excitation light source 1008, a sample well member 1010 including a first sample well region 1006A and a second sample well region 1006B, and a fluorescence imaging device 1002 which may be configured as described herein including in connection with FIGS. 1 and 3A-3C. For instance, the first sample well region 1006A and the second sample well regions 1006B may include respective sample wells configured to support respective portions of a sample.

In some embodiments, the fluorescence imaging device 1002 may be configured to direct an imaging aperture 1004 thereof toward a sample well region 1006 and, after directing the imaging aperture 1004 toward the sample well region 1006, capture an image of the sample well region 1006 using the imaging aperture 1004. For example, the imaging aperture 1004 of the fluorescence imaging device 1002 may be defined as described herein including in connection with FIG. 3C. In the illustrated embodiment, the fluorescence imaging device 1002 may be configured to direct the imaging aperture 1004 toward the first sample well region 1006A, then capture a first image of the first sample well region 1006A using the imaging aperture 1004, then redirect the imaging aperture 1004 toward the second sample well region 1006B, and then capture a second image of the second sample well region 1006B using the imaging aperture 1004.

In some embodiments, an image may include an electrical signal produced over time from fluorescent light emitted following a single excitation of the sample, and/or may include electrical signals reflecting an aggregation of fluorescent light emitted following a plurality of excitations of the sample. For example, when very little fluorescence is expected to be emitted following an excitation, aggregating fluorescent emissions over a plurality of excitations may produce an image with high intensity, making the resulting image easier to process in some applications, though aggregation need not be performed in all cases.

In some embodiments, the fluorescence imaging device 1002 may be configured to optically and/or mechanically direct the imaging aperture 1004 toward a sample well region 1006. For example, the fluorescence imaging device 1002 may include a mechanical scanner configured to move the fluorescence imaging device 1002 relative to the first sample well region 1006A to direct the imaging aperture 1004 toward the first sample well region 1006A and/or to move the fluorescence imaging device 1002 relative to the second sample well region 1006B to redirect the imaging aperture 1004 toward the second sample well region 1006B. For instance, as shown in FIG. 10, the fluorescence imaging device 1002 may be movable (e.g., using a mechanical scanner) in a scanning direction such that the imaging aperture 1004 of the fluorescence imaging device 1002 may move correspondingly along the scanning direction from being directed toward the first sample well region 1006A as shown in FIG. 10 to being directed toward the second sample well region 1006B (see, e.g., FIG. 3B). While the fluorescence imaging device 1002 is shown in FIG. 10 being movable in the scanning direction, it should be appreciated that the sample well member 1010 may be alternatively or additionally movable in the scanning direction such that the fluorescence imaging device 1002 and the sample well regions 1006 are moved relative to one another.

In the same or another example, the fluorescence imaging device 1002 may include an optical scanner configured to optically steer the imaging aperture 1004 in a direction of the first sample well region to direct the imaging aperture 1004 toward the first sample well region 1006A and optically steer the imaging aperture 1004 in a direction of the second sample well 1006B region to redirect the imaging aperture 1004 toward the second sample well region 1006B. For instance, as shown in FIG. 10, the fluorescence imaging device 1002 may be configured to steer the imaging aperture 1004 (e.g., using an optical scanner) about a steering axis, such as from being directed toward the first sample well region 1006A as shown in FIG. 10 to being redirected toward the second sample well region 1006B (see, e.g., FIG. 3B). While a single steering axis is shown in FIG. 10, steering may be implemented using multiple axes (e.g., azimuth and elevation) depending on the application.

In some embodiments, the excitation light source 1008 may be configured to illuminate the first sample well region 1006A to excite the first portion of the sample to emit fluorescent light that the fluorescence imaging device 1002 is configured to capture in the first image and illuminate the second sample well region 1006B to excite the second portion of the sample to emit fluorescent light that the fluorescence imaging device 1002 is configured to capture in the second image. For example, as shown in FIG. 10, the excitation light source 1008 may be configured to illuminate a sample well region 1006 toward which the imaging aperture 1004 of the fluorescence imaging device 1002 is directed. For instance, the fluorescence imaging device 1002 and the excitation light source 1008 may be controlled using a same control circuit 110, such as described herein including in connection with FIG. 1. Alternatively or additionally, the excitation light source 1008 may be configured to illuminate multiple or all sample well regions 1006 even though the imaging aperture 1004 of the fluorescence imaging device 1002 may be directed toward fewer (e.g., one) sample well regions 1006 than are illuminated with excitation light.

Though not shown in FIG. 10, in some embodiments the fluorescence imaging system 1000 may further include a processing device configured to determine fluorescence information of the sample using a first image of the first sample well region 1006A and a second image of the second sample well region 1006B, such as described herein including in connection with FIG. 1. For example, the fluorescence information may include individual fluorescence information obtained from a respective image of the first image and the second image, and/or may include combined fluorescence information obtained from the first image and the second image combined.

In some embodiments, the fluorescence imaging device 1002 may be further configured to redirect the imaging aperture 1004 toward the first sample well region 1006A after capturing the second image of the second sample well region 1006B. For example, the fluorescence imaging device 1002 may be configured to alternate between capturing an image of the first sample well region 1006A and capturing an image of the second sample well region 1006B (e.g., following controlled cleavage in the respective sample well region).

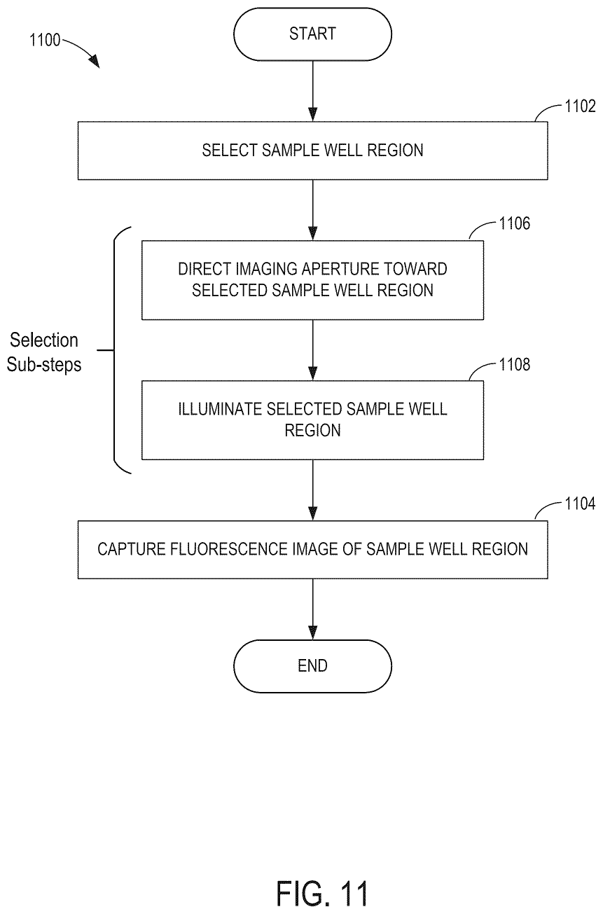

FIG. 11 is a flow diagram of an example fluorescence imaging method 1100 including selecting a sample well region and capturing an image of the sample well region, according to some embodiments.

As shown in FIG. 11, the method includes a step 1102 of selecting a sample well region (e.g., from among a first sample well region and a second sample well region) and a step 1104 of capturing a fluorescence image of the selected sample well region. In some embodiments, the method 1100 of FIG. 11 may be performed using the fluorescence imaging system of any one of FIG. 1, FIGS. 3A-3C, and/or FIG. 10. For example, the steps 1102, 1104 of selecting a sample well region and/or capturing the fluorescence image may be performed using the fluorescence imaging device 1002 of FIG. 10.

In some embodiments, the step 1102 of selecting a sample well region 304 may be performed as described herein including in connection with FIGS. 3A-3C. For example, a fluorescence imaging device 108 may select and capture a first image of a first sample well 304A region and/or a second image of a second sample well region 304B. For instance, a first image of a first sample well region 304A may not include at least a portion of a second sample well region 304B and/or a second image of a second sample well region 304B may not include at least a portion of a first sample well region 304A.

As further shown in FIG. 11, the step 1102 of selecting a sample well region includes a sub-step 1106 of directing an imaging aperture towards the selected sample well region and a sub-step 1108 of illuminating the selected sample well region. For example, the sub-step of directing the imaging aperture 1004 towards the selected sample well region 1006 and the sub-step of illuminating the selected sample well region 1006 may be performed as described herein including in connection with FIG. 10. For instance, the sub-step 1106 of directing the imaging aperture 1004 towards the selected sample well region 1006 may include moving, (e.g., by a mechanical scanner) the fluorescence imaging device 1002 relative to the selected sample well region 1006 and/or optically steering (e.g., by an optical scanner) the imaging aperture 1004 in a direction of the selected sample well region 1006.

In some embodiments, the fluorescence imaging device 1002 may direct the imaging aperture 1004 toward the selected sample well region 1006 (e.g., without being directed towards at least one other sample well region) and an excitation light source 1008 may illuminate the selected sample well region 1006 (and in some cases other sample well regions 1006 as well).

While not shown in FIG. 11, it should be appreciated that the method 1100 may further include a step of, by a processing device (e.g., of the system 102 of FIG. 1), determining fluorescence information of the sample using the image (e.g., using a first image and a second image of a first sample well region and of a second sample well region, respectively).

In some embodiments, the step 1102 of selecting the sample well region may include selecting the first sample well region as the sample well region, and the step 1104 of capturing the fluorescence image may include capturing a first image of the first sample well region, and the method may further include a step of selecting a second sample well region as the sample well region and capturing a second image of the second sample well region (not shown). For example, the method 1100 may include a step 1106 of directing an imaging aperture of a fluorescence imaging device toward a first sample well region 1006A, then a step 1104 of capturing, using the imaging aperture 1004, a first image of the first sample well region 1006A, then a step of redirecting the imaging aperture 1004 toward the second sample well region 1006B, and then a step 1104 of capturing, using the imaging aperture 1004, a second image of the second sample well region 1006B, such as described herein including in connection with FIG. 10. In some embodiments, further steps may be performed in the method 1100 to repetitively capture images of the sample well regions, such as following steps of controlled cleavage in the sample well regions, respectively, as described herein.

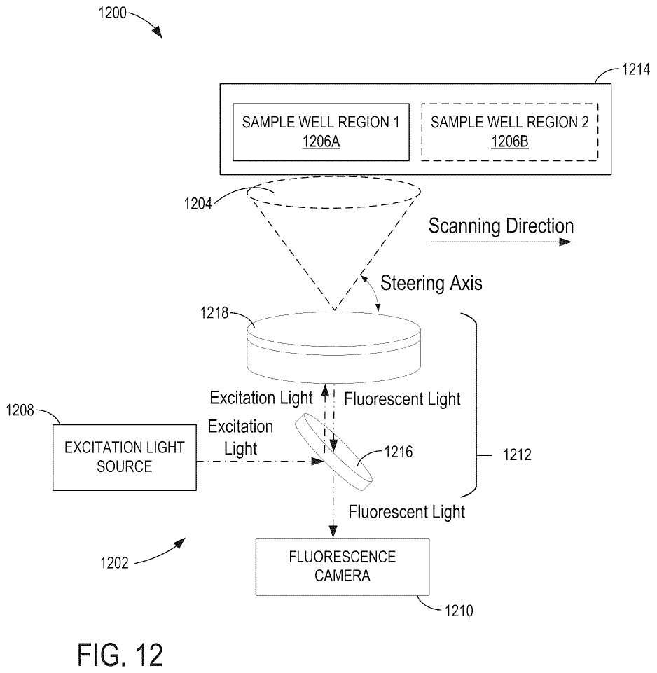

FIG. 12 is an optical diagram of an alternative example fluorescence imaging system 1200 including a fluorescence imaging device 1202 configured to direct an imaging aperture 1204 towards a sample well region 1206 and capture an image of the sample well region 1206 using the imaging aperture 1204, according to some embodiments.

In some embodiments, the fluorescence imaging system of FIG. 12 may be configured as described herein for the system 1000 of FIG. 10 and may be used to perform any methods which may be performed using the system 1200 of FIG. 12. For example, as shown in FIG. 12, the fluorescence imaging system includes a fluorescence imaging device and a sample well member including a first sample well region 1206A and a second sample well region 1206B, which may be configured as described herein including in connection with FIG. 1, FIGS. 3A-3C, and FIG. 10. In the illustrated embodiment, the fluorescence imaging device 1202 includes an excitation light source 1208, which may be configured as described herein including in connection with FIG. 1, FIGS. 3A-3C, and FIG. 10.

As further shown in FIG. 12, the fluorescence imaging device 1202 includes a fluorescence camera 1210 and optical components 1212 optically coupled between the fluorescence camera 1210, the excitation light source 1208, and the sample well member 1214. For example, the optical components 1212 may be configured to deliver excitation light to the sample well member 1214 and to deliver fluorescent light from the sample well member 1214 to the fluorescence camera 1210. For instance, as shown in FIG. 12, the optical components 1212 include a beamsplitter 1216 and an objective lens 1218 which may be configured to direct excitation light from the excitation light source 1208 to the sample well member 1214 and to deliver fluorescent light from the sample well member 1214 to the fluorescence camera 1210. In the illustrated embodiment, the objective lens 1218 (e.g., and/or other optical components 1212) of the fluorescence imaging device 1202 may be configured to optically and/or mechanically steer (e.g., along a scanning direction and/or about a steering axis, respectively) the imaging aperture 1204 of the fluorescence imaging device 1202, such as described herein including in connection with FIG. 10. In some embodiments, the fluorescence camera 1210 may include a photodetector pixel array, such as described herein for the fluorescence imaging device 108 of FIG. 1.

In some embodiments, the beamsplitter 1216 may be configured as a dichroic beamsplitter, such as having a cutoff wavelength between a wavelength of excitation light the excitation light source 1208 is configured to emit and a wavelength of fluorescent light the fluorescence camera 1210 is configured to capture. In some embodiments, the objective lens 1218 may have a narrow bandwidth (e.g., shorter than for full-color imaging), such as including a range of excitation light wavelengths and a range of fluorescent light wavelengths and not including, for example, at least some visible light wavelength ranges.

In some embodiments, the excitation light source 1208, fluorescence camera 1210, and optical components 1212 may be housed together (e.g., in a same housing). For example, the housing may have one or more ports configured to interface with the sample well region(s) 1206 of the sample well member 1214.

FIG. 13 is an optical flow diagram of an alternative example fluorescence imaging system 1300 including a fluorescence imaging device 1302 including a plurality of fluorescence cameras 1304, according to some embodiments.

In some embodiments, the fluorescence imaging system 1300 of FIG. 13 may be configured as described herein including in connection with FIG. 12. For example, the fluorescence imaging system 1300 includes a sample well member 1214 including multiple sample well regions 1206 and an excitation light source 1208, and the fluorescence imaging device 1302 includes optical components 1306 coupled to the excitation light source 1208. In the illustrated example, the fluorescence imaging device 1302 includes multiple fluorescence cameras 1304 configured to receive fluorescent light emitted from the sample well region 1206 toward which the imaging aperture 1204 is directed.

In some embodiments, a fluorescence imaging system 1300 may include a fluorescence imaging device 1302 configured to capture fluorescent light emitted from a sample, the fluorescent light comprising a first portion having content contained within a first optical band 1308A and a second portion having content contained within a second optical band 1308B. For example, as shown in FIG. 13, the fluorescence imaging device 1302 includes a first fluorescence camera 1304A (“Camera 1”), which may be configured to capture light contained within the first optical band 1308A, and a second fluorescence camera 1304B (“Camera 2”), which may be configured to capture light contained within a second optical band 1308B. For instance, the first fluorescence camera 1304A and the second fluorescence cameras 1304B may be configured to generate electrical signals only for light received in the respective optical bands 1308, and/or may be configured to filter out light received outside of the respective optical bands. In some embodiments, the first optical band 1308A and the second optical band 1308B may be disjoint, whereas in other embodiments the first optical band 1308A and the second optical band 1308B may overlap at least in part.

While the fluorescence imaging device 1302 includes multiple fluorescence cameras 1304, it should be appreciated that a single fluorescence camera may be configured to capture portions of fluorescent light contained within respective optical bands 1308, such as described herein including in connection with FIG. 12. It should also be appreciated that, in some embodiments, the fluorescence cameras 1304 shown in FIG. 13 may be configured to receive light in the same optical band or bands 1308, as embodiments described herein are not so limited.

In some embodiments, a fluorescence imaging device 1302 may include optical components 1306 configured to receive and provide fluorescent light to first and second fluorescence cameras 1304A, 1304B, the fluorescent light comprising a first portion having content contained within the first optical band 1308A provided to the first fluorescence camera 1304A and a second portion having content contained within the second optical band 1308B provided to the second fluorescence camera 1304B. For example, some of the optical components 1306 may be configured to receive the fluorescent light, such as the objective lens 1218 and the first beam splitter 1310A, coupled to the excitation light source 1208. For instance, the objective lens 1218 and the first beam splitter 1310A may be configured to receive and transmit light in both optical bands 1308 therethrough to the fluorescence cameras 1304. Alternatively or additionally, some of the optical components 1306 may be configured to divide the fluorescent light into the first portion of the fluorescent light and the second portion of the fluorescent light, such as the second beam splitter 1310B shown coupled between the fluorescence cameras 1304. For instance, the second beam splitter 1310B may be configured to transmit light in the first optical band 1308A (“Band 1”) to the first fluorescence camera 1304A and to refract light in the second optical band 1308B (“Band 2”) to the second fluorescence camera 1304B.

In some embodiments, the second beam splitter 1310B may include a dichroic mirror having a cutoff wavelength between, at least in part, the first optical band and the second optical band. For example, the cutoff wavelength may be entirely between the optical bands 1308 where the bands are disjoint, and/or may be between non-overlapping portions of the optical bands 1308 where the bands at least partially overlap.

In some embodiments, optical components 1306 of the fluorescence imaging device 1302 may be configured to divide the received fluorescent light between a first optical path that includes the first fluorescence camera 1304A and a second optical path that includes the second fluorescence camera 1304B. For example, in FIG. 13, the second beam splitter 1310B is optically coupled to the first optical path that includes (e.g., terminates at) the first fluorescence camera 1304A and further optically coupled to the second optical path that includes (e.g., terminates at) the second fluorescence camera 1304B. While no other optical elements 1306 are shown in FIG. 13 in the optical paths between the second beam splitter and the fluorescence cameras, respectively, it should be appreciated that other components such as tube lenses and/or associated tip and/or tilt adjustment mechanisms may be present.

As shown in FIG. 13, the fluorescence imaging system 1300 includes an excitation light source 1208 configured to illuminate the sample with excitation light to excite the sample to emit the fluorescent light. In other embodiments, a fluorescence imaging device 1302 may not include an excitation light source, as embodiments described herein are not so limited.

In some embodiments, processing circuitry 112 (e.g., FIG. 1) of the system 1300 may be configured to determine fluorescence information of the sample based on a relationship between intensity of the first portion of the fluorescent light and intensity of the second portion of the fluorescent light. For example, as described further herein in connection with FIGS. 14A-14B, a sample may be characterized by a relationship between spectral content in a first optical band 1308A and in a second optical band 1308B. In some embodiments, characterizing a sample (e.g., discriminating between fluorescent light emitted by recognition molecules) using a relationship between spectral content in a first optical band 1308A and in a second optical band 1308B may permit low cost and/or scalable fluorescence imaging devices 1302 to be used, such as by removing the need for determining fluorescence lifetime information by discriminating fluorescent light based on arrival time. It should be appreciated that lifetime information and/or discrimination based on arrival time may be alternatively or additionally used as embodiments described herein are not so limited.

FIG. 14A is a plot 1400 of normalized fluorescent light intensity vs. ratio of intensity of a portion of fluorescent light in a first optical band with respect to intensity of a portion of fluorescent light in a second optical band, according to some embodiments.

In some embodiments, fluorescence information of a sample may be determined by processing circuitry 112 (e.g., of a system as in FIG. 1), the fluorescence information comprising a relationship between intensity of a first portion of fluorescent light emitted by the sample and intensity of a second portion of the fluorescent light emitted by the sample. For example, the first portion of the fluorescent light may include content contained within a first optical band and the second portion of the fluorescent light may include content contained within a second optical band. For instance, the intensities of the content in the respective bands may indicate spectral content of the fluorescent light, which in turn may be used to characterize the sample.

In some embodiments, a relationship between intensities of portions of fluorescent light emitted by a sample may be used to discriminate between recognition molecules in the sample. For example, the sample may include multiple recognition molecules 1402 configured to emit fluorescent light having spectral content in one or each of the portions of the fluorescent light. For instance, the relationship between intensity of the first portion (e.g., contained within a first optical band) and intensity of the second portion (e.g., contained within a second optical band) may indicate which of multiple recognition molecules 1402 emitted the fluorescent light, which in turn may indicate which recognition molecule 1402 has bound to the sample, which in turn may identify a portion (e.g., a terminal amino acid) of (e.g., a polypeptide of) the sample.

According to various embodiments, the processing circuitry 112 may be configured to determine a ratio of the intensity of the first portion of the fluorescent light with respect to the intensity of the second portion of the fluorescent light, a proportion of the intensity of the first portion of the fluorescent light, a proportion of the intensity of the second portion of the fluorescent light, that the relationship between the intensities of the portions of the fluorescent light associates the fluorescent light with a constrained vector space.

FIG. 14A illustrates an example of a ratio of intensity of a first portion of fluorescent light with respect to intensity of a second portion of the fluorescent light. In the illustrated example, the sample under test includes 6 recognition molecules 1402 labeled PS1165, PS1220, PS1259, PS610, PS2132, and PS1223, each of which may emit fluorescent light in a distinct region of intensity vs. ratio. For example, PS2132 is shown having content substantially in only a first optical band 1308A (“passthrough,” e.g., Band 1 in FIG. 13) and PS1220 is shown having content substantially in only a second optical band 1308B (“reflected,” e.g. Band 2 in FIG. 13), which may serve to distinguish the two recognition molecules from one another and from the other recognition molecules 1402 shown in FIG. 14A. As another example, PS1165 and PS1223 are shown having substantially similar intensities, and may be distinguished by PS1223 having a higher ratio than PS1165, such as due to differing center wavelengths of their associated fluorescent emissions. In some embodiments, intensity may serve as a further basis for distinguishing recognition molecules 1402 in addition to ratio. For example, PS1259 and PS1165 are shown having similar ratios, and may be distinguished by PS1165 having a higher intensity than PS1259. According to various embodiments, processing circuitry 112 described herein may be configured to discriminate among recognition molecules 1402 based on individual measurements, and/or based on statistical groupings of measurements collected during a predetermined time period (e.g., after dispensing a terminal amino acid modifier and/or catalyst for removal of a terminal amino acid). For example, the groupings of measurements may be from a time during which only one recognition molecule 1402 is expected to bind to the sample and emit fluorescent light.

In alternative or additional embodiments, the recognition molecules 1402 of the illustrated example may be distinguished by a proportion of intensity of the first portion of the fluorescent light and/or a proportion of the second portion of the fluorescent light. For example, PS2132 may be distinguished from PS1220 by a higher proportion of intensity in the first optical band 1308A and a lower proportion of intensity in the second optical band 1308B. A proportion of intensity of a first portion of the fluorescent light may be determined as a ratio of the intensity of the first portion of the fluorescent light with respect to a sum of the first portion and the second portion of the fluorescent light.

In further alternative or additional embodiments, the recognition molecules 1402 of the illustrated example may be distinguished by association of intensities of the first portion and the second portion of the fluorescent light with a constrained vector space. For example, the first portion and the second portion of the fluorescent light may be included as vector components of a vector, which may be plotted in a vector space (e.g., 2D vector space) of intensity of the first portion (e.g., in the first optical band) vs. intensity of the second portion (e.g., in the second optical band). For instance, the intensity of the first portion and the intensity of the second portion may place the resulting vector in a cluster within a predetermined vector distance of other vectors associated with a recognition molecule. In the illustrated example, the first portion and the second portion corresponding to PS2132 may cluster together in 2D vector space with vector components of intensity of the first portion and the second portion of the fluorescent light, respectively, such as with the vector space satisfying a constraint that the intensity of the first portion is significantly higher than the intensity of the second portion. It should be appreciated that vector constraints may be imposed using a predetermined threshold vector distance and/or using a vector distance determined to statistically link the intensities of the first portion and the second portion with respective recognition molecules.

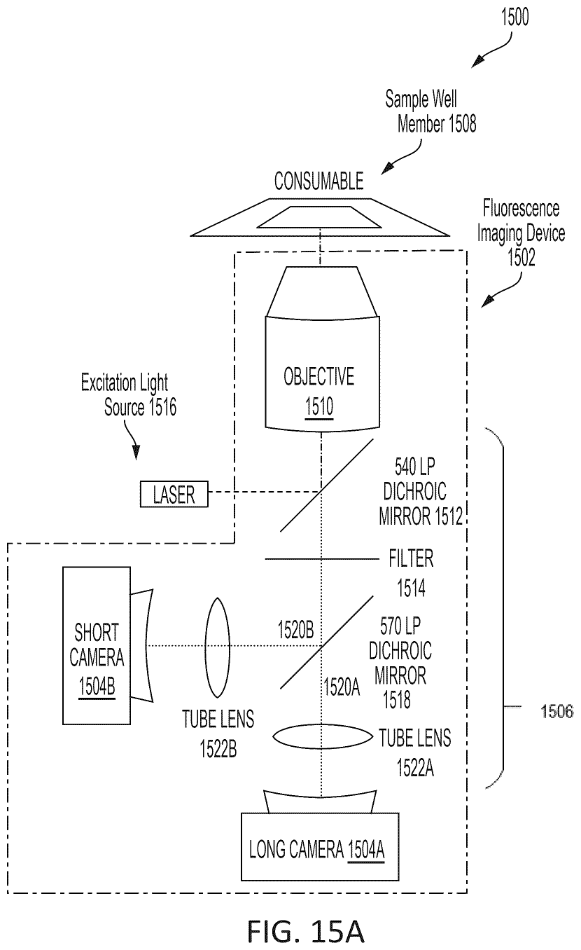

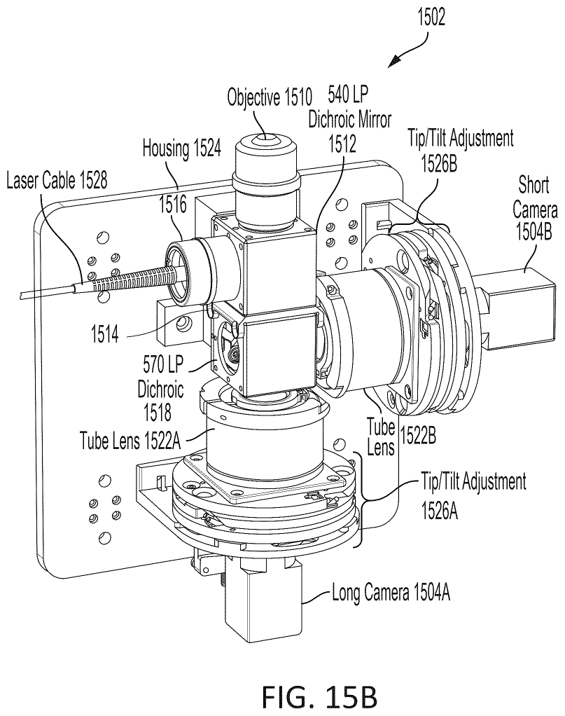

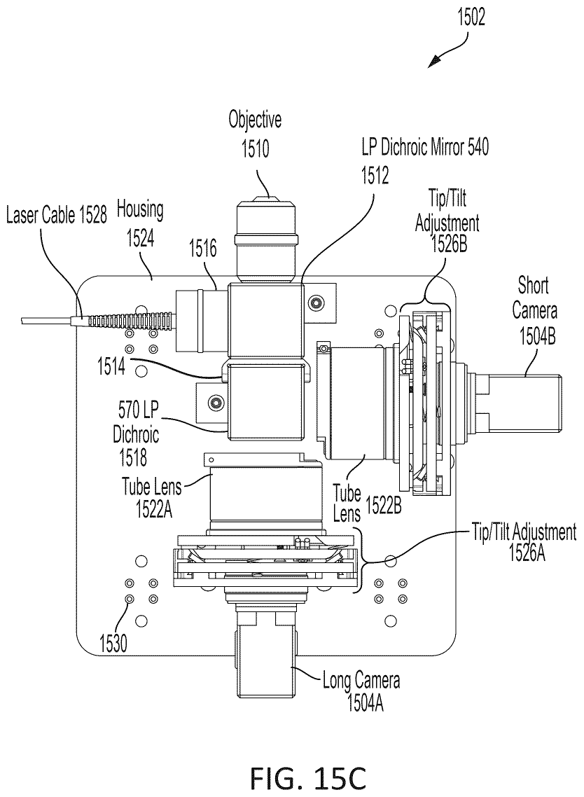

In some embodiments, fluorescence information of a sample may be determined (e.g., by processing circuitry as in FIG. 1) based on a first image, generated by a first fluorescence camera capturing a first portion of fluorescent light, emitted by the sample, and a second image, generated by a second fluorescence camera capturing a second portion of fluorescent light emitted by the sample. For example, as described herein including in connection with FIGS. 15A-15C, a fluorescence imaging device may include multiple fluorescence cameras. For instance, the fluorescence cameras may be configured to receive light having contained in respective optical bands.

In some embodiments, one or more recognition molecules may emit fluorescent light having spectral content in each of a first optical band and a second optical band. For example, while some fluorescence imaging devices may include a camera for each recognition molecule (e.g., configured to receive light in an optical band associated with emissions from the respective recognition molecule), other fluorescence imaging devices may include fewer cameras than recognition molecules between which the fluorescence imaging device may be configured to discriminate. For instance, a relationship between intensity of fluorescent light contained within a first optical band and intensity of fluorescent light contained within a second optical band may indicate a recognition molecule that emits some content in the first optical band and some content in the second optical band, or which emits content only in one of the first optical band and the second optical band.

FIG. 14B is a plot 1420 of fluorescent light intensity vs. ratio of intensity of a portion of fluorescent light in a first optical band with respect to intensity of a portion of the fluorescent light in a second optical band, according to some embodiments.

In FIG. 14B, fluorescent light intensity combined from multiple optical bands is shown in non-normalized distribution and plotted against spectral ratio in the manner described herein in connection with FIG. 16A.

In the illustrated example of FIG. 14B, the sample may include 4 recognition molecules labeled recognizers (“Recog.”) 1, 2, 3, and 4, which of which may emit fluorescent light in a distinct region of spectral ratio. For example, recognizers 1, 2, 3, and 4 are shown in FIG. 14B having increasing spectral ratios compared to one another, which may serve to distinguish the recognizers from one another. In some embodiments, processing circuitry 112 described herein may be configured to discriminate among recognition molecules 1402 as described herein including in connection with FIG. 14A.

It should be appreciated that the recognition molecules 1402 in the illustrated example of FIG. 14B may be alternatively or additionally distinguished using other relationships between intensity of respective portions of the fluorescent light such as described herein including in connection with FIG. 14A.

FIG. 14C is a plot 1430 of fluorescent light intensity vs. color ratio of a portion of fluorescent light in a first optical band 1308A with respect to intensity of a portion of the fluorescent light in a second optical band 1308B for a plurality of fluorescently tagged DNA species, according to some embodiments.

The plurality of fluorescently tagged DNA species include DNA structures with at least one small molecule fluorescent die conjugated to the DNA structures using methods known in the art. In the example shown, the plurality of fluorescently tagged DNA species includes 4Cy3B (DNA structures to which four Cy3B molecules are conjugated), C2C (DNA structures to which two Cy3 molecules and one Cy3B molecules are conjugated), C6C (DNA structures to which six Cy3 molecules and one iFluor 570 molecule is attached), 4C530NS (DNA structures to which four C530NS molecules are attached), 4ATR6G (DNA structures to which four ATTO Rho6G molecules are attached), 3BGN (DNA structures to which three BDP3037 molecules attached), and 4GGN (DNA structures to which four BDP3014 molecules are attached) see, e.g., PCT International Publication No.: WO2025/147654A1, filed Jan. 3, 2025, the contents of which are incorporated herein by reference in their entirety. As shown, the fluorescence intensity of an exemplary FRET dye attached to duplex DNA is shown. Each of the plurality of fluorescently tagged DNA species may emit fluorescent light in a distinct region of spectral ratio. For example, each of the fluorescently tagged DNA species have different intensities and color ratios, as shown in FIG. 14B, which may serve to distinguish the fluorescently tagged DNA species from one another. In some embodiments, processing circuitry 112 described herein may be configured to discriminate among the plurality of fluorescently tagged DNA species as described herein including in connection with FIG. 14A.

It should be appreciated that the plurality of fluorescently tagged DNA species in the illustrated example of FIG. 14C may be alternatively or additionally distinguished using other relationships between intensity of respective portions of the fluorescent light such as described herein including in connection with FIG. 14A.

FIG. 15A is an optical diagram 1500 of an example fluorescence imaging device 1502 including a plurality of fluorescence cameras 1504, according to some embodiments.

In some embodiments, the fluorescence imaging device 1502 shown in FIG. 15A may be configured as described herein for the fluorescence imaging device 1302 of FIG. 13. For example, as shown in FIG. 15A, the fluorescence imaging device 1502 includes optical components 1506 configured to receive fluorescent light emitted by a sample, such as may be supported by the illustrated sample well member 1508 shown as a consumable in FIG. 15A. For instance, the optical components 1506 in FIG. 15A include an objective lens 1510, a first dichroic mirror 1512 configured as a long-pass (LP) filter, and an optical filter 1514. In the illustrated embodiment, the first dichroic mirror 1512 has a cutoff wavelength of 540 nanometers (nm), which may transmit fluorescent light and refract excitation light from the excitation light source 1516, such as in embodiments in which excitation light has a shorter wavelength than fluorescent light.

Also shown in FIG. 15A, the fluorescence imaging device 1502 includes a first fluorescence camera 1504A (“Long Camera”) and a second fluorescence camera 1504B (“Short Camera”) configured to receive respective portions of the fluorescent light. For example, the optical components 1506 further include a second dichroic mirror configured as an LP filter coupled between the first and second fluorescence cameras. For instance, in FIG. 15A, the second dichroic mirror 1518 has a cutoff wavelength of 570 nm, which may be configured to divide the fluorescent light between light contained within a first optical band 1520A and light contained within a second optical band 1520B. Also shown in FIG. 15A, the second dichroic mirror 1518 is configured to divide the fluorescent light into a first optical path, including the first fluorescence camera 1504A and a first intervening tube lens 1522A, and a second optical path, including the second fluorescent camera 1504B and a second intervening tube lens 1522B.

FIG. 15B is a perspective view of an example fluorescence imaging device 1502 implemented based on the optical diagram 1500 of FIG. 15A, according to some embodiments. FIG. 15C is a side view of the fluorescence imaging device 1502 of FIG. 15B, according to some embodiments.

As shown in FIGS. 15B-15C, the fluorescence imaging device of FIG. 15A is implemented together with a housing 1524 that holds the optical components 1506 relative to the fluorescence cameras 1504, and with tip and/or tilt adjustment mechanisms 1526 for the tube lenses 1522 interposed between the fluorescence cameras 1504 and respective tube lenses 1522. For example, the tip and/or tilt adjustment mechanisms 1526 may be controlled (e.g., by a controller as described herein) to adjust a position of the respective fluorescence camera 1504 with respect to a sample well (e.g., sample well region), such as described herein. In the illustrated embodiment, the excitation light source 1516 is coupled to the first dichroic mirror 1512 by a cable 1528 (e.g., a fiber optic cable).

In some embodiments, the housing 1524 may be configured to be driven by a motor (e.g., of the fluorescence imaging system) to scan an imaging aperture of the fluorescence imaging device 1502 among a plurality of sample well regions for selective imaging. For example, as shown in FIGS. 15B-15B, the housing includes holes 1530 that may be configured for mounting (e.g., via fixtures such as screws) to a movable bracket controlled by a motor.

It should be appreciated that the cutoff wavelengths used in FIGS. 15A-15C are examples and that other cutoff wavelengths may be used depending on the implementation.

FIG. 16A is a cross-section of a first integrated device 1600 of a fluorescence imaging system containing a sample well member 1602 and a fluorescence imaging device 1604, according to some embodiments. FIG. 16B is a cross-section of an alternative integrated device 1620 of a fluorescence imaging system containing a sample well member 1622 and a fluorescence imaging device 1624, respectively, according to some embodiments.

In some embodiments, the integrated devices 1600, 1620 shown in FIGS. 16A-16B may be included in the fluorescence imaging system of any one or each of FIG. 1, FIGS. 3A-3C, and/or FIG. 10. For example, the illustrated sample well member 1602, 1622 and fluorescence imaging device 1604, 1624 may be configured as described herein for the sample well member and fluorescence imaging device of any one or each of FIG. 1, FIGS. 3A-3C, and FIG. 10. For instance, the sample well member 1602, 1622 may include a plurality of sample well regions configured to support respective portions of a sample, and the fluorescence imaging device may 1604, 1624 be configured to capture fluorescent light emitted by the sample. While not shown in FIGS. 16A-16B, it should be appreciated that in some embodiments the integrated device containing the fluorescence imaging device 1604, 1624 may be configured to move and/or optically scan relative to the integrated device containing the sample well member, such as described herein including in connection with FIG. 10.

As shown in FIGS. 16A-16B, the illustrated integrated devices 1600, 1620 include a light coupling region 1-201, a light propagation region 1-202, and a pixel region 1-203. For example, the light coupling region 1-201 may be configured to receive excitation light from an excitation light source (not shown), which the light propagation region 1-202 may be configured to propagate to the pixel region 1-203 for exciting a sample supported by the sample well member. For instance, in FIGS. 16A-16B, the light coupling region 1-201 includes a grating coupler 1-216 that may be configured to receive and optically couple excitation light to the light propagation region 1-202, and the light propagation region 1-202 includes a waveguide 1-220 that may be configured to couple excitation light from the grating coupler 1-216 to the pixel region 1-203. While a single grating coupler 1-216 and a single waveguide 1-220 are shown in FIGS. 16A-16B, it should be appreciated that multiple grating couplers may be included and/or a network of waveguides may be included, such as to receive and propagate excitation light to an array of sample wells within one or more sample regions.

In some embodiments, the pixel region 1-203 may include sample wells 1-108 arranged within sample well regions of the sample well member, which may be configured to receive excitation light from the waveguide 1-220. For example, the waveguide 1-220 may be configured to evanescently couple excitation light to the sample wells 1-108 to distribute the excitation light among the sample wells 1-108. In the illustrated embodiment, the pixel region 1-203 further includes a metal layer 1-106 that may be configured to shield the layers below from light external to the illustrated portions of the integrated devices.

In some embodiments, the pixel region 1-203 may further include an array of photodetector pixels 1-110 configured to capture fluorescent light emitted from one or more corresponding sample wells 1-108 of the sample well member. For example, in FIGS. 16A-16B, photodetector pixels 1-110 are shown aligned (e.g., along an optica direction OPT) with respective sample wells 1-108, which may cause fluorescent light emitted by the sample wells 1-108 to reach photodetection regions 1-112 of the photodetector pixels 1-110. In the illustrated embodiment, metal layers 1-240 are further present proximate (e.g., between) photodetector pixels 1-110, such as to carry control signals to the photodetector pixels 1-110 (e.g., to control a timing of capturing fluorescence images). Also shown in FIGS. 16A-16B, the fluorescence imaging device includes optical layers 1-230, which may include optical components configured to direct fluorescent light from the sample wells 1-108 toward the photodetection regions 1-112 and/or to direct excitation light (e.g., leaked from the waveguide 1-220) away from the photodetection regions 1-112, such as to improve the ratio of captured fluorescent light with respect to captured excitation light.

In some embodiments, the integrated devices shown in FIGS. 16A-16B may be configured for removably coupling to one another. For example, the integrated device containing the sample well member 1602, 1622 may be configured to be consumed from supporting a sample, and thus may be replaced with another sample well member 1602, 1622. In the example of FIG. 16A, the integrated device containing the sample well member 1602 may further include the grating coupler 1-216 and/or waveguide 1-220, whereas in the example of FIG. 16B, the grating coupler 1-216 and/or waveguide 1-220 may be included in the integrated device containing the fluorescence imaging device 1624. For instance, the example of FIG. 16A may be advantageous for applications in which it is desirable to use an integrated device including only a fluorescence imaging device 1604 (e.g., without optical components for providing excitation light to the sample well member) whereas the example of FIG. 16B may be advantageous for applications in which it is desirable to use a sample well member 1602 including only sample wells (e.g., without optical components for providing excitation light to the sample wells), though either type of example system may be used depending on the particular application.

In some embodiments, the fluorescence imaging devices shown in FIGS. 16A-16B may be configured as the fluorescence camera 1210 of the system 1200 of FIG. 12, such as with the optical components 1212 shown in FIG. 12 optically coupling the fluorescence camera 1210 to the excitation light source 1208 and to the sample well member 1214, which may not contain a grating coupler and waveguides as shown in the examples of FIGS. 16A-16B.

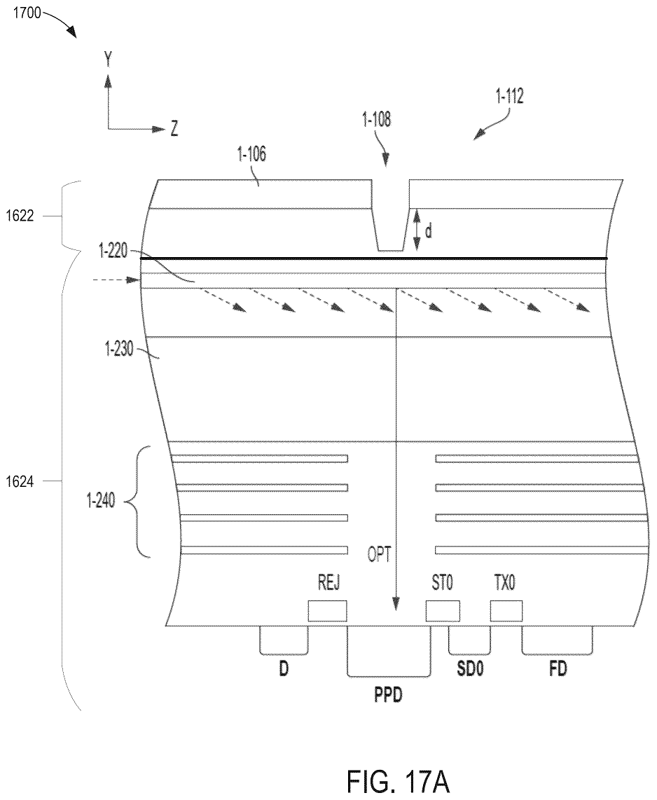

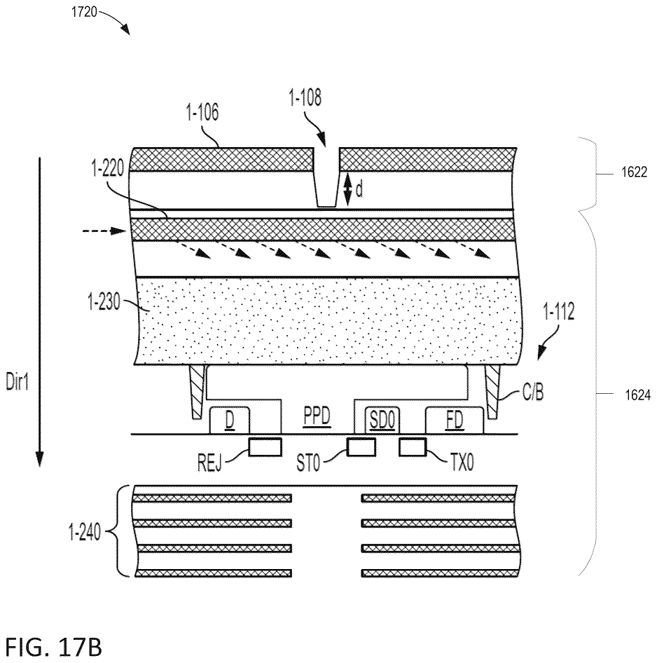

FIG. 17A is a magnified cross-section of a portion of the integrated devices of FIG. 16B, according to some embodiments. FIG. 17B is a magnified cross-section of a portion of an alternative integrated device that may be included in the system of FIG. 16B, according to some embodiments.

As shown in FIGS. 17A-17B, a single sample well 1-108 is located above a waveguide 1-220 and further above a photodetector pixel including a photodetection region PPD, a rejection region D, a charge storage region SD0, a readout (e.g., floating diffusion) region FD, and transfer gates REJ, ST0, and TX0. In some embodiments, transfer gates REJ, ST0, and TX0 (e.g., polysilicon and/or metal-oxide-semiconductor gates) may be configured to receive control signals via the metal layers 1-240 to control operation of the photodetector pixel.

In some embodiments, the photodetector pixel may be configured to capture light received from the sample well region including the sample well 1-108 to generate a fluorescence image. For example, fluorescent light emitted from the sample well 1-108 may reach the photodetection region PPD (e.g., a pinned photodiode) causing generation of charge carriers (e.g., photoelectrons) therein. In some embodiments, during illumination of the sample well 1-108 using the excitation light source, the transfer gate REJ may be activated to transfer charge carriers generated in the photodetection region PPD to the rejection region REJ. In some embodiments, following illumination of the sample well 1-108 and/or during emission of fluorescent light from the sample well 1-108, the transfer gate ST0 may be configured to transfer charge carriers generated in the photodetection region PPD to the charge storage region SD0 (e.g., a storage diode). In some embodiments, following one or more periods of receiving fluorescent light, generating charge carriers, and storing the charge carriers in the charge storage region SD0, the transfer gate TX0 may be configured to transfer charge carriers stored and/or aggregated (e.g., over multiple excitations of the sample well) to the readout region FD to be read out (e.g., as an electrical signal indicating the received fluorescent light).

FIG. 17A illustrates a front-side illumination (FSI) configuration 1700 in which the transfer gates REJ, ST0, and TX0 are on a side of the fluorescence imaging device 1624 that faces the sample wells in a sample well region, whereas FIG. 17B illustrates a backside illumination (BSI) configuration 1720 in which the photodetection region PPD is on a first side of the fluorescence imaging device 1624 facing the sample wells in a sample well region and the transfer gates REJ, ST0, and TX0 are on an opposite side of the fluorescence imaging device 1624 away from the sample wells. In FIG. 17B, charged and/or biased (C/B) regions are positioned around the photodetection region PPD (e.g., between the illustrated pixel and adjacent pixels of the array), which may be configured to induce charge carrier depletion in the photodetection region PPD.

In some embodiments, pixels of the fluorescence imaging device 1624 may be configured to capture light received from respective sample well regions to generate respective images. For example, the pixels illustrated in FIGS. 17A-17B may be configured to capture light using the photodetection region PPD and charge storage region SD0, and to generate an image at least in part by reading out a signal indicative of received charge via the readout region FD. In some embodiments, pixels of the fluorescence imaging device 1624 may be configured to discriminate between capturing and rejecting received light based upon a wavelength of the light and/or based upon a time at which the light arrives at the array of pixels. For example, wavelength-based discrimination may be achieved using photodetection regions of different depths and/or having optical filters with different wavelength passbands above the photodetection regions, respectively, such that charge carriers generated by the photodetection regions and stored in the same or respective charge storage regions may correspond to different wavelengths of received light. In the same or another example, arrival time-based discrimination may be achieved by controlling charge carrier transfer to the rejection region D or to the charge storage region SD0 depending on the time at which the charge carriers are generated in the photodetection region, such that charge carriers stored in the charge storage region (and/or in respective charge storage regions) correspond to an arrival time (and/or respective arrival times) of received light.

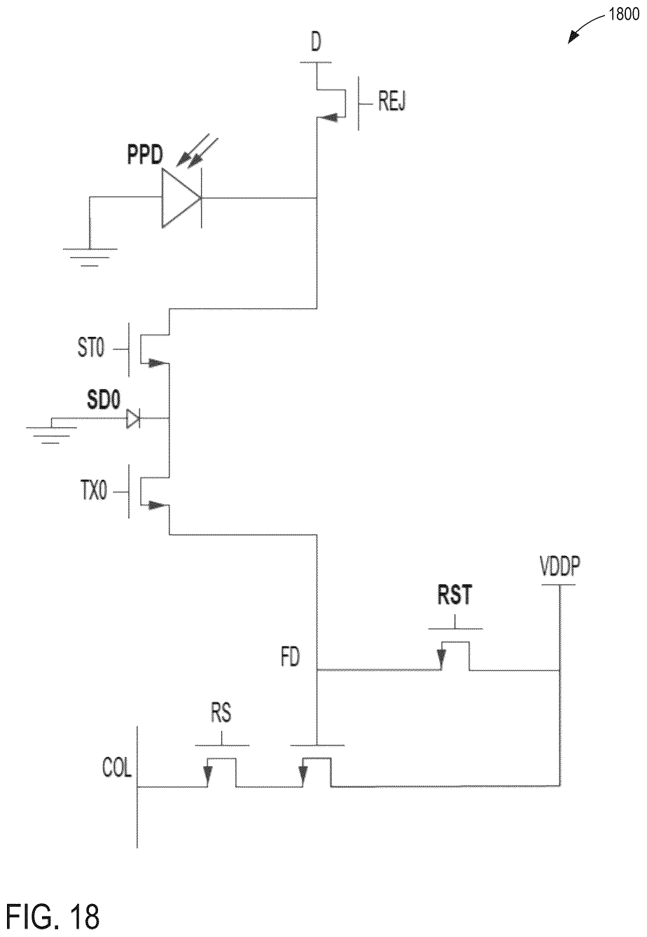

FIG. 18 is a circuit diagram of an example pixel circuit 1800 that may be included in the fluorescence imaging device 1604 of FIG. 16A, according to some embodiments.

In some embodiments, the pixel circuit of FIG. 18 may be configured as described herein for the photodetector pixel of FIG. 17A. For example, the transfer gates REJ, ST0, and TX0 may be configured as gates of transistors in the pixel circuit, with the rejection region being coupled to a power supply voltage, the photodetection region PPD and charge storage region SD0 biased with a ground connection, and with the readout region FD being coupled to the gate of a buffer transistor coupled to a column readout line (COL, e.g., for a column of photodetector pixels), and a reset (RST) transistor being coupled to the readout region FD to control applying a power supply voltage VDDP to the readout region FD.

Having thus described several aspects and embodiments of the technology of the present disclosure, it is to be appreciated that various alterations, modifications, and improvements will readily occur to those of ordinary skill in the art. Such alterations, modifications, and improvements are intended to be within the spirit and scope of the technology described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments may be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media may be transportable, such that the program or programs stored thereon may be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that may be employed to program a computer or other processor to implement various aspects as described above. Additionally, it should be appreciated that according to one aspect, one or more computer programs that when executed perform methods of the present disclosure need not reside on a single computer or processor, but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code may be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more input and output devices. These devices may be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The terms “substantially,” “approximately,” and “about” may be used to mean within ±20% of a target value in some embodiments, within ±10% of a target value in some embodiments, within ±5% of a target value in some embodiments, and yet within ±2% of a target value in some embodiments. The terms “substantially,” “approximately,” and “about” may include the target value.

1. A fluorescence imaging system, comprising:
a first sample well region comprising a first sample well configured to support a first portion of a sample;
a second sample well region comprising a second sample well configured to support a second portion of the sample; and
a fluorescence imaging device configured to:
(a) direct an imaging aperture of the fluorescence imaging device toward the first sample well region;
(b) after (a), capture, using the imaging aperture, a first image of the first sample well region;
(c) after (b), redirect the imaging aperture toward the second sample well region; and
(d) after (c), capture, using the imaging aperture, a second image of the second sample well region.

2. The fluorescence imaging system of claim 1, further comprising a processing device configured to determine fluorescence information of the sample using the first image and the second image.

3. The fluorescence imaging system of claim 2, wherein:
the fluorescence imaging device comprises a first fluorescence camera configured to receive a first portion of fluorescent light using the imaging aperture and a second fluorescence camera configured to receive a second portion of fluorescent light using the imaging aperture;
the first image comprises the first portion of the fluorescent light and the second portion of the fluorescent light; and
the processing device is configured to determine the fluorescence information of the sample using a relationship between intensity of the first portion of the fluorescent light and intensity of the second portion of the fluorescent light.

4. The fluorescence imaging system of claim 3, wherein the relationship comprises a ratio of the intensity of the first portion of the fluorescent light with respect to the intensity of the second portion of the fluorescent light.

5. The fluorescence imaging system of claim 3, wherein the relationship comprises a proportion of the intensity of the first portion of the fluorescent light and/or a proportion of the intensity of the second portion of the fluorescent light.

6. The fluorescence imaging system of claim 3, wherein the processing device is configured to determine that the relationship associates the intensity of the first portion of the fluorescent light and the intensity of the second portion of the fluorescent light with a constrained vector space.

7. The fluorescence imaging system of any one of claims 1 to 6, wherein the fluorescence imaging device comprises a mechanical scanner configured to move the fluorescence imaging device relative to the first sample well region to perform (a) and move the fluorescence imaging device relative to the second sample well region to perform (c).

8. The fluorescence imaging system of any one of claims 1 to 7, wherein the fluorescence imaging device comprises an optical scanner configured to optically steer the imaging aperture in a direction of the first sample well region to perform (a) and optically steer the imaging aperture in a direction of the second sample well region to perform (c).

9. The fluorescence imaging system of any one of claims 1 to 8, wherein the imaging aperture is smaller than a combined area of the first sample well region and the second sample well region.

10. The fluorescence imaging system of claim 9, wherein the fluorescence imaging device is configured to capture light in a first direction, the imaging aperture is in a first plane that is transverse to the first direction, and the combined area of the first sample well region and the second sample well region is in a second plane parallel to the first plane.

11. The fluorescence imaging system of claim 9 or 10, further comprising:
a member including the first sample well region and the second sample well region,
wherein the imaging aperture is smaller than an area of the member that comprises the first sample well region and the second sample well region.

12. The fluorescence imaging system of any one of claims 1 to 11, wherein the first sample well region and the second sample well region are included within a consumable member that is configured to be consumed from supporting the sample.

13. The fluorescence imaging system of any one of claims 1 to 12, further comprising an excitation light source configured to:
illuminate the first sample well region to excite the first portion of the sample to emit fluorescent light that the fluorescence imaging device is configured to capture in the first image; and
illuminate the second sample well region to excite the second portion of the sample to emit fluorescent light that the fluorescence imaging device is configured to capture in the second image.

14. The fluorescence imaging system of any one of claims 1 to 13, wherein the fluorescence imaging device comprises an integrated circuit comprising an array of pixels configured to capture light received from the first sample well region to generate the first image and to capture light received from the second sample well region to generate the second image, wherein the imaging aperture is at least an area of the array of pixels.

15. The fluorescence imaging system of claim 14, wherein pixels of the array of pixels are configured to discriminate between received light based upon a wavelength of the light and/or based upon a time at which the light arrives at the array of pixels.

16. The fluorescence imaging system of any one of claims 1 to 15, wherein the first sample well region comprises:
a first plurality of sample wells comprising the first sample well and configured to support the first portion of the sample; and
a second plurality of sample wells comprising the second sample well and configured to support the second portion of the sample.

17. A method of performing fluorescence imaging of a sample using a fluorescence imaging device, the sample having a first sample portion supported by a first sample well of a first sample well region and a second sample portion supported by a second sample well of a second sample well region, and the method comprising:
(a) directing an imaging aperture of the fluorescence imaging device toward the first sample well region;
(b) after (a), capturing, using the imaging aperture, a first image of the first sample well region;
(c) after (b), redirecting the imaging aperture toward the second sample well region; and
(d) after (c), capturing, using the imaging aperture, a second image of the second sample well region.

18. The method of claim 17, further comprising, by a processing device, determining fluorescence information of the sample using the first image and the second image.

19. The method of claim 18, wherein:
(b) comprises receiving, by a first fluorescence camera of the fluorescence imaging device, using the imaging aperture, a first portion of fluorescent light and receiving, by a second fluorescence camera of the fluorescence imaging device, using the imaging aperture, a second portion of fluorescent light;
the first image comprises the first portion of the fluorescent light and the second portion of the fluorescent light; and
determining the fluorescence information of the sample comprises using a relationship between intensity of the first portion of the fluorescent light and intensity of the second portion of the fluorescent light.

20. The method of claim 19, wherein the relationship comprises a ratio of the intensity of the first portion of the fluorescent light with respect to the intensity of the second portion of the fluorescent light.

21. The method of claim 19, wherein the relationship comprises a proportion of the intensity of the first portion of the fluorescent light and/or a proportion of the intensity of the second portion of the fluorescent light.

22. The method of claim 19, wherein determining the fluorescence information of the sample comprises determining that the relationship associates the intensity of the first portion of the fluorescent light and the intensity of the second portion of the fluorescent light with a constrained vector space.

23. The method of any one of claims 17 to 22, wherein:
(a) comprises moving, by a mechanical scanner, the fluorescence imaging device relative to the first sample well region; and
(c) comprises moving, by the mechanical scanner, the fluorescence imaging device relative to the second sample well region.

24. The method of any one of claims 17 to 23, wherein:
(a) comprises optically steering, by an optical scanner, the imaging aperture in a direction of the first sample well region; and
(c) comprises optically steering, by the optical scanner, the imaging aperture in a direction of the second sample well region.

25. The method of any one of claims 17 to 24, wherein the imaging aperture is smaller than a combined area of the first sample well region and the second sample well region.

26. The method of claim 25, wherein the fluorescence imaging device captures light in a first direction, the imaging aperture is in a first plane that is transverse to the first direction, and the combined area of the first sample well region and the second sample well region is in a second plane parallel to the first plane.

27. The method of claim 25 or 26, wherein:
a member includes the first sample well region and the second sample well region; and
the imaging aperture is smaller than an area of the member that comprises the first sample well region and the second sample well region.

28. The method of any one of claims 17 to 27, wherein the first sample well region and the second sample well region are included within a consumable member that is consumed from supporting the sample.

29. The method of any one of claims 17 to 28, further comprising:
illuminating, by an excitation light source, the first sample well region to excite the first sample portion to emit fluorescent light that the fluorescence imaging device captures in the first image; and
illuminating, by the excitation light source, the second sample well region to excite the second sample portion to emit fluorescent light that the fluorescence imaging device is configured to capture in the second image.

30. The method of any one of claims 17 to 29, wherein:
(b) comprises capturing, by an array of pixels of an integrated circuit, light received from the first sample well region to generate the first image;
(d) comprises capturing, by the array of pixels, light received from the second sample well region to generate the second image; and
the imaging aperture is at least an area of the array of pixels.

31. The method of claim 30, wherein each of (b) and (d) comprises pixels of the array of pixels discriminating between received light based upon a wavelength of the light and/or based upon a time at which the light arrives at the array of pixels.

32. The method of any one of claims 17 to 31, wherein the first sample well region comprises:
a first plurality of sample wells comprising the first sample well and supporting the first sample portion; and
a second plurality of sample wells comprising the second sample well and supporting the second sample portion.

33. A fluorescence imaging system, comprising:
a first sample well region comprising a first sample well configured to support a first portion of a sample;
a second sample well region comprising a second sample well configured to support a second portion of the sample; and
a fluorescence imaging device configured to:
select a sample well region from among the first sample well region and the second sample well region; and
capture an image of the sample well region.

34. The fluorescence imaging system of claim 33, further comprising a processing device configured to determine fluorescence information of the sample using the image.

35. The fluorescence imaging system of claim 34, wherein:
the fluorescence imaging device comprises a first fluorescence camera configured to receive a first portion of fluorescent light and a second fluorescence camera configured to receive a second portion of fluorescent light;
the image comprises the first portion of the fluorescent light and the second portion of the fluorescent light; and
the processing device is configured to determine the fluorescence information of the sample using a relationship between intensity of the first portion of the fluorescent light and intensity of the second portion of the fluorescent light.

36. The fluorescence imaging system of claim 35, wherein the relationship comprises a ratio of intensity of the first portion of the fluorescent light with respect to intensity of the second portion of the fluorescent light.

37. The fluorescence imaging system of claim 35, wherein the relationship comprises a proportion of the intensity of the first portion of the fluorescent light and/or a proportion of the intensity of the second portion of the fluorescent light.

38. The fluorescence imaging system of claim 35, wherein the processing device is configured to determine that the relationship associates the intensity of the first portion of the fluorescent light and the intensity of the second portion of the fluorescent light with a constrained vector space.

39. The fluorescence imaging system of any one of claims 33 to 38, wherein the fluorescence imaging device is configured to:
select the first sample well region as the sample well region;
capture a first image of the first sample well region;
select the second sample well region as the sample well region; and
capture a second image of the second sample well region.

40. The fluorescence imaging system of claim 39, wherein the first image does not include at least a portion of the second sample well region and the second image does not include at least a portion of the first sample well region.

41. The fluorescence imaging system of claim 39 or 40, further comprising a processing device configured to determine fluorescence information of the sample using the first image and the second image.

42. The fluorescence imaging system of any one of claims 33 to 41, wherein the fluorescence imaging device is configured to capture the image of the sample well region using an imaging aperture that is smaller than a combined area of the first sample well region and the second sample well region.

43. The fluorescence imaging system of claim 42, wherein the fluorescence imaging device is configured to capture light in a first direction, the imaging aperture is in a first plane that is transverse to the first direction, and the combined area of the first sample well region and the second sample well region is in a second plane parallel to the first plane.

44. The fluorescence imaging system of claim 43, further comprising:
a member including the first sample well region and the second sample well region,
wherein the imaging aperture is smaller than an area of the member that comprises the first sample well region and the second sample well region.

45. The fluorescence imaging system of any one of claims 33 to 44, wherein the first sample well region and the second sample well region are included within a consumable member that is configured to be consumed from supporting the sample.

46. The fluorescence imaging system of any one of claims 33 to 45, further comprising an excitation light source configured to:
illuminate the sample well region to excite a portion of the sample to emit fluorescent light that the fluorescence imaging device is configured to capture in the image.

47. The fluorescence imaging system of any one of claims 33 to 46, wherein the fluorescence imaging device comprises an integrated circuit comprising an array of pixels configured to capture light received from the sample well region to generate the image.

48. The fluorescence imaging system of claim 47, wherein pixels of the array of pixels are configured to discriminate between received light based upon a wavelength of the light and/or based upon a time at which the light arrives at the array of pixels.

49. The fluorescence imaging system of any one of claims 33 to 48, wherein the first sample well region comprises:
a first plurality of sample wells comprising the first sample well and configured to support the first portion of the sample; and
a second plurality of sample wells comprising the second sample well and configured to support the second portion of the sample.

50. A method of performing fluorescence imaging of a sample using a fluorescence imaging device, the sample having a first sample portion supported by a first sample well of a first sample well region and a second sample portion supported by a second sample well of a second sample well region, and the method comprising:
selecting, by a fluorescence imaging device, a sample well region from among the first sample well region and the second sample well region; and
capturing, by the fluorescence imaging device, an image of the sample well region.

51. The method of claim 50, further comprising, by a processing device, determining fluorescence information of the sample using the image.

52. The method of claim 51, wherein:
capturing the image comprises receiving, by a first fluorescence camera of the fluorescence imaging device, a first portion of fluorescent light and receiving, by a second fluorescence camera of the fluorescence imaging device, a second portion of fluorescent light;
the image comprises the first portion of the fluorescent light and the second portion of the fluorescent light; and
determining the fluorescence information of the sample comprises using a relationship between intensity of the first portion of the fluorescent light and intensity of the second portion of the fluorescent light.

53. The method of claim 52, wherein the relationship comprises a ratio of the intensity of the first portion of the fluorescent light with respect to the intensity of the second portion of the fluorescent light.

54. The method of claim 52, wherein the relationship comprises a proportion of the intensity of the first portion of the fluorescent light and/or a proportion of the intensity of the second portion of the fluorescent light.

55. The method of claim 52, wherein determining the fluorescence information of the sample comprises determining that the relationship associates the intensity of the first portion of the fluorescent light and the intensity of the second portion of the fluorescent light with a constrained vector space.

56. The method of any one of claims 50 to 55, wherein:
selecting the sample well region comprises selecting the first sample well region as the sample well region;
capturing the image comprises capturing a first image of the first sample well region;
the method further comprises selecting, by the fluorescence imaging device, the second sample well region as the sample well region; and
the method further comprises capturing a second image of the second sample well region.

57. The method of claim 56, wherein the first image does not include at least a portion of the second sample well region and the second image does not include at least a portion of the first sample well region.

58. The method of claim 56 or 57, further comprising, by a processing device, determining fluorescence information of the sample using the first image and the second image.

59. The method of any one of claims 50 to 58, wherein capturing the image uses an imaging aperture that is smaller than a combined area of the first sample well region and the second sample well region.

60. The method of claim 59, wherein the fluorescence imaging device is configured to capture light in a first direction, the imaging aperture is in a first plane that is transverse to the first direction, and the combined area of the first sample well region and the second sample well region is in a second plane parallel to the first plane.

61. The method of claim 60, wherein:
a member includes the first sample well region and the second sample well region; and
the imaging aperture is smaller than an area of the member that comprises the first sample well region and the second sample well region.

62. The method of any one of claims 50 to 61, wherein the first sample well region and the second sample well region are included within a consumable member that is consumed from supporting the sample.

63. The method of any one of claims 50 to 62, further comprising:
illuminating, by an excitation light source, the sample well region to excite a portion of the sample to emit fluorescent light that the fluorescence imaging device captures in the image.

64. The method of any one of claims 50 to 63, wherein capturing the image comprises capturing, by an array of pixels of an integrated circuit, light received from the sample well region to generate the image.

65. The method of claim 64, wherein capturing the image comprises pixels of the array of pixels discriminating between received light based upon a wavelength of the light and/or based upon a time at which the light arrives at the array of pixels.

66. The method of any one of claims 50 to 65, wherein the first sample well region comprises:
a first plurality of sample wells comprising the first sample well and configured to support the first sample portion; and
a second plurality of sample wells comprising the second sample well and configured to support the second sample portion.

67. A method of polypeptide sequencing, the method comprising:
(a) capturing, by a fluorescence imaging device, a fluorescence image of a first sample well; and
(b) in a second sample well, performing a controlled cleavage of a terminal amino acid of a polypeptide immobilized to a surface of the second sample well, the controlled cleavage comprising catalyzing removal of the terminal amino acid of the polypeptide,
wherein (a) is performed during at least a portion of (b).

68. The method of claim 67, wherein:
(b) further comprises modifying the terminal amino acid of the polypeptide prior to catalyzing removal of the terminal amino acid; and
catalyzing the removal of the terminal amino acid uses an enzyme having increased catalytic activity for removal of the terminal amino acid following modification with respect to before the modification.

69. The method of claim 67 or 68, wherein (a) further comprises contacting a polypeptide immobilized to a surface of the first sample well with a composition comprising one or more terminal amino acid recognition molecules.

70. The method of claim 69, wherein (a) further comprises illuminating the first sample well with an excitation light source and capturing fluorescent light emitted by the first sample well using the fluorescence imaging device.

71. The method of any one of claims 67 to 70, further comprising:
(c) following (b), capturing a fluorescence image of the second sample well.

72. The method of claim 71, wherein (c) further comprises contacting the polypeptide immobilized to the surface of the second sample well with a composition comprising one or more terminal amino acid recognition molecules.

73. The method of any one of claims 67 to 72, wherein:
wherein the first sample well is included in a first sample well region and the second sample well is included in a second sample well region; and
the fluorescence imaging device has an imaging aperture that is smaller than a combined area of the first sample well region and the second sample well region.

74. The method of claim 73, wherein the fluorescence imaging device captures light in a first direction, the imaging aperture is in a first plane that is transverse to the first direction, and the combined area of the first sample well region and the second sample well region is in a second plane parallel to the first plane.

75. The method of claim 73 or 74, wherein:
a member includes the first sample well region and the second sample well region; and
the imaging aperture is smaller than an area of the member that comprises the first sample well region and the second sample well region.

76. The method of any one of claims 73 to 75, wherein the first sample well region comprises:
a first plurality of sample wells comprising the first sample well; and
a second plurality of sample wells comprising the second sample well.

77. The method of any one of claims 67 to 76, wherein the first sample well and the second sample well are included within a consumable member that is consumed from supporting a sample.

78. The method of any one of claims 67 to 77, wherein:
(a) comprises capturing, by an array of pixels of an integrated circuit of the fluorescence imaging device, light received from the first sample well to generate the fluorescence image.

79. The method of claim 78, wherein (a) comprises pixels of the array of pixels discriminating between received light based upon a wavelength of the light and/or based upon a time at which the light arrives at the array of pixels.

80. The method of any one of claims 67 to 79, wherein:
(a) comprises receiving, by a first fluorescence camera of the fluorescence imaging device, a first portion of fluorescent light and receiving, by a second fluorescence camera of the fluorescence imaging device, a second portion of fluorescent light;
the fluorescence image comprises the first portion of the fluorescent light and the second portion of the fluorescent light; and
the method further comprises (d) determining, by a processing device, fluorescence information of the polypeptide, and (d) comprises using a relationship between intensity of the first portion of the fluorescent light and intensity of the second portion of the fluorescent light.

81. The method of claim 80, wherein the relationship comprises a ratio of the intensity of the first portion of the fluorescent light with respect to the intensity of the second portion of the fluorescent light.

82. The method of claim 80, wherein the relationship comprises a proportion of the intensity of the first portion of the fluorescent light and/or a proportion of the intensity of the second portion of the fluorescent light.

83. The method of claim 80, wherein (d) comprises determining that the relationship associates the intensity of the first portion of the fluorescent light and the intensity of the second portion of the fluorescent light with a constrained vector space.

84. The method of claim any one of claims 80 to 83, wherein (d) comprises identifying a terminal amino acid recognition molecule from which the fluorescent light was emitted.

85. A fluorescence imaging system, comprising:
a fluorescence imaging device configured to capture a fluorescence image of a sample well region located within an imaging aperture of the fluorescence imaging device over a duration of imaging; and
a control circuit configured to set the duration of imaging and/or a location of the imaging aperture.

86. The fluorescence imaging system of claim 85, further comprising a user interface device, wherein the control circuit is configured to set the duration of imaging and/or the location of the imaging aperture based on instructions received via the user interface device.

87. The fluorescence imaging system of claim 86, wherein the instructions indicate a selected type of measurement to be performed by the fluorescence imaging system, and the duration of imaging and/or the location of the imaging aperture are based on the selected type of measurement.

88. The fluorescence imaging system of any one of claims 85 to 87, wherein the control circuit is configured to set a plurality of locations of the imaging aperture corresponding to a plurality of sample well regions, respectively.

89. The fluorescence imaging system of claim 88, wherein the control circuit is further configured to set the duration of imaging including a plurality of sub-durations of imaging the plurality of sample well regions, respectively.

90. The fluorescence imaging system of claim 89, wherein the plurality of sub-durations of imaging the plurality of sample well regions comprise different sub-durations.

91. The fluorescence imaging system of any one of claims 85 to 90, wherein the control circuit is configured to set the duration of imaging based on a selected type of sequencing measurement of a plurality of predetermined types of sequencing measurements.

92. The fluorescence imaging system of claim 91, wherein the control circuit is configured to set the duration of imaging in a range from a first duration based on a first predetermined type of sequencing measurement to a second duration based on a second predetermined type of sequencing measurement.

93. The fluorescence imaging system of claim 91 or 92, wherein the plurality of predetermined types of sequencing measurements are selected from a group consisting of:
sequencing of a predetermined polypeptide;
sequencing using only wavelength-based recognition molecule discrimination; and
sequencing using at least time-based recognition molecule discrimination.

94. The fluorescence imaging system of any one of claims 85 to 93, further comprising a processing device configured to determine fluorescence information of a sample in the sample well region using the fluorescence image.

95. The fluorescence imaging system of claim 94, wherein:
the fluorescence imaging device comprises a first fluorescence camera configured to receive a first portion of fluorescent light using the imaging aperture and a second fluorescence camera configured to receive a second portion of fluorescent light using the imaging aperture;
the fluorescence image comprises the first portion of the fluorescent light and the second portion of the fluorescent light; and
the processing device is configured to determine the fluorescence information of the sample using a relationship between intensity of the first portion of the fluorescent light and intensity of the second portion of the fluorescent light.

96. The fluorescence imaging system of claim 95, wherein the relationship comprises a ratio of intensity of the first portion of the fluorescent light with respect to intensity of the second portion of the fluorescent light.

97. The fluorescence imaging system of claim 95, wherein the relationship comprises a proportion of the intensity of the first portion of the fluorescent light and/or a proportion of the intensity of the second portion of the fluorescent light.

98. The fluorescence imaging system of claim 95, wherein the processing device is configured to determine that the relationship associates the intensity of the first portion of the fluorescent light and the intensity of the second portion of the fluorescent light with a constrained vector space.

99. The fluorescence imaging system of any one of claims 85 to 98, further comprising a plurality of sample well regions including the sample well region, wherein the imaging aperture is smaller than a combined area of the plurality of sample well regions.

100. The fluorescence imaging system of claim 99, wherein the fluorescence imaging device is configured to capture light in a first direction, the imaging aperture is in a first plane that is transverse to the first direction, and the combined area of the plurality of sample well regions is in a second plane parallel to the first plane.

101. The fluorescence imaging system of claim 99 or 100, further comprising:
a member including the plurality of sample well regions,
wherein the imaging aperture is smaller than an area of the member that comprises the plurality of sample well regions.

102. The fluorescence imaging system of any one of claims 85 to 101, wherein the sample well region is included within a consumable member that is configured to be consumed from supporting a sample.

103. The fluorescence imaging system of any one of claims 85 to 102, further comprising an excitation light source configured to:
illuminate the sample well region to excite a portion of a sample to emit fluorescent light that the fluorescence imaging device is configured to capture in the fluorescence image.

104. The fluorescence imaging system of any one of claims 85 to 103, wherein the fluorescence imaging device comprises an integrated circuit comprising an array of pixels configured to capture light received from the sample well region to generate the fluorescence image.

105. The fluorescence imaging system of claim 104, wherein pixels of the array of pixels are configured to discriminate between capturing and rejecting received light based upon a wavelength of the light and/or based upon a time at which the light arrives at the array of pixels.

106. A method of performing fluorescence imaging of a sample using a fluorescence imaging device, the sample being located in a sample well region during the fluorescence imaging, and the method comprising:
setting, by a control circuit, a duration of imaging and/or a location of an imaging aperture of the fluorescence imaging device; and
capturing, by the fluorescence imaging device, over the duration of imaging, while the sample well region is located within the imaging aperture of the fluorescence imaging device, a fluorescence image of the sample well region.

107. The method of claim 106, further comprising receiving instructions via a user interface device, wherein setting the duration of imaging and/or the location of the imaging aperture is based on the instructions.

108. The method of claim 107, wherein the instructions indicate a selected type of measurement to be performed, and the duration of imaging and/or the location of the imaging aperture are set based on the selected type of measurement.

109. The method of any one of claims 106 to 108, wherein the control circuit sets a plurality of locations of the imaging aperture corresponding to a plurality of sample well regions, respectively.

110. The method of claim 109, wherein the control circuit further sets the duration of imaging including a plurality of sub-durations of imaging the plurality of sample well regions, respectively.

111. The method of claim 110, wherein the plurality of sub-durations of imaging the plurality of sample well regions comprise different sub-durations.

112. The method of any one of claims 106 to 111, wherein setting the duration of imaging is based on a selected type of sequencing measurement of a plurality of predetermined types of sequencing measurements.

113. The method of claim 112, wherein setting the duration of imaging is in a range from a first duration based on a first predetermined type of sequencing measurement to a second duration based on a second predetermined type of sequencing measurement.

114. The method of claim 112 or 113, wherein the plurality of predetermined types of sequencing measurements are selected from a group consisting of:
sequencing of a predetermined polypeptide;
sequencing using only wavelength-based recognition molecule discrimination; and
sequencing using at least time-based recognition molecule discrimination.

115. The method of any one of claims 106 to 114, further comprising determining, by a processing device, fluorescence information of a sample in the sample well region using the fluorescence image.

116. The method of claim 115, wherein:
capturing the fluorescence image comprises receiving, by a first fluorescence camera of the fluorescence imaging device, using the imaging aperture, a first portion of fluorescent light and receiving, by a second fluorescence camera of the fluorescence imaging device, using the imaging aperture, a second portion of fluorescent light;
the fluorescence image comprises the first portion of the fluorescent light and the second portion of the fluorescent light; and
determining the fluorescence information of the sample comprises using a relationship between intensity of the first portion of the fluorescent light and intensity of the second portion of the fluorescent light.

117. The method of claim 116, wherein the relationship comprises a ratio of the intensity of the first portion of the fluorescent light with respect to the intensity of the second portion of the fluorescent light.

118. The method of claim 116, wherein the relationship comprises a proportion of the intensity of the first portion of the fluorescent light and/or a proportion of the intensity of the second portion of the fluorescent light.

119. The method of claim 116, wherein determining the fluorescence information of the sample comprises determining that the relationship associates the intensity of the first portion of the fluorescent light and the intensity of the second portion of the fluorescent light with a constrained vector space.

120. The method of any one of claims 106 to 119, wherein a plurality of sample well regions include the sample well region, wherein the imaging aperture is smaller than a combined area of the plurality of sample well regions.

121. The method of claim 120, wherein capturing the fluorescence image comprises capturing light in a first direction, the imaging aperture is in a first plane that is transverse to the first direction, and the combined area of the plurality of sample well regions is in a second plane parallel to the first plane.

122. The method of claim 120 or 121, wherein:
a member includes the plurality of sample well regions; and
the imaging aperture is smaller than an area of the member that comprises the plurality of sample well regions.

123. The method of any one of claims 106 to 122, wherein the sample well region is included within a consumable member that is configured to be consumed from supporting a sample.

124. The method of any one of claims 106 to 123, further comprising:
illuminating, by an excitation light source, the sample well region to excite a portion of the sample to emit fluorescent light that the fluorescence imaging device captures in the fluorescence image.

125. The method of any one of claims 106 to 124, wherein capturing the fluorescence image comprises capturing, by an array of pixels of an integrated circuit, light received from the sample well region to generate the fluorescence image.

126. The method of claim 125, wherein capturing the fluorescence image comprises discriminating, by pixels of the array of pixels, between capturing and rejecting received light based upon a wavelength of the light and/or based upon a time at which the light arrives at the array of pixels.

127. A fluorescence imaging device, comprising:
a first fluorescence camera configured to capture light contained within a first optical band;
a second fluorescence camera configured to capture light contained within a second optical band; and
optical components configured to:
receive fluorescent light emitted by a sample, the fluorescent light comprising a first portion having content contained within the first optical band and a second portion having content contained within the second optical band;
provide the first portion of the fluorescent light to the first fluorescence camera; and
provide the second portion of the fluorescent light to the second fluorescence camera.

128. The fluorescence imaging device of claim 127, wherein the optical components are further configured to divide the fluorescent light into the first portion of the fluorescent light and the second portion of the fluorescent light.

129. The fluorescence imaging device of claim 127 or 128, wherein the optical components comprise a dichroic mirror having a cutoff wavelength between the first optical band and the second optical band.

130. A fluorescence imaging system comprising:
the fluorescence imaging device of any one of claims 127 to 129; and
processing circuitry configured to determine fluorescence information of the sample, the fluorescence information comprising and/or based on a relationship between intensity of the first portion of the fluorescent light and intensity of the second portion of the fluorescent light.

131. The fluorescence imaging system of claim 130, wherein the relationship comprises a ratio of the intensity of the first portion of the fluorescent light with respect to the intensity of the second portion of the fluorescent light.

132. The fluorescence imaging system of claim 130, wherein the relationship comprises a proportion of the intensity of the first portion of the fluorescent light and/or a proportion of the intensity of the second portion of the fluorescent light.

133. The fluorescence imaging system of claim 130, wherein the processing circuitry is configured to determine that the relationship associates the intensity of the first portion of the fluorescent light and the intensity of the second portion of the fluorescent light with a constrained vector space.

134. The fluorescence imaging system of any one of claims 130 to 133, wherein the processing circuitry is configured to determine the fluorescence information of the sample based on a first image, generated by the first fluorescence camera capturing the first portion of the fluorescent light, and a second image, generated by the second fluorescence camera capturing the second portion of the fluorescent light.

135. The fluorescence imaging system of any one of claims 130 to 134, wherein the processing circuitry is configured to discriminate between a first recognition molecule and a second recognition molecule in the sample based on the relationship.

136. The fluorescence imaging system of claim 135 wherein first recognition molecule and/or the second recognition molecule emits fluorescent light having spectral content in each of the first optical band and the second optical band.

137. The fluorescence imaging system of any one of claims 130 to 136, further comprising an excitation light source configured to illuminate the sample with excitation light to excite the sample to emit the fluorescent light.

138. A fluorescence imaging system, comprising:
a fluorescence imaging device configured to capture fluorescent light emitted from a sample, the fluorescent light comprising a first portion having content contained within a first optical band and a second portion having content contained within a second optical band; and
processing circuitry configured to determine fluorescence information of the sample based on a relationship between intensity of the first portion of the fluorescent light and intensity of the second portion of the fluorescent light.

139. The fluorescence imaging system of claim 138, wherein the relationship comprises a ratio of the intensity of the first portion of the fluorescent light with respect to the intensity of the second portion of the fluorescent light.

140. The fluorescence imaging system of claim 138, wherein the relationship comprises a proportion of the intensity of the first portion of the fluorescent light and/or a proportion of the intensity of the second portion of the fluorescent light.

141. The fluorescence imaging system of claim 138, wherein the processing circuitry is configured to determine that the relationship associates the intensity of the first portion of the fluorescent light and the intensity of the second portion of the fluorescent light with a constrained vector space.

142. The fluorescence imaging system of any one of claims 138 to 141, wherein the fluorescence imaging device comprises a first fluorescence camera configured to capture the first portion of the fluorescent light and a second fluorescence camera configured to capture the first portion of the fluorescent light.

143. The fluorescence imaging system of claim 142, wherein the processing circuitry is configured to determine the fluorescence information of the sample based on a first image, generated by the first fluorescence camera capturing the first portion of the fluorescent light, and a second image, generated by the second fluorescence camera capturing the second portion of the fluorescent light.

144. The fluorescence imaging system of claim 143, wherein the processing circuitry is configured to discriminate between a first recognition molecule and a second recognition molecule in the sample based on the relationship.

145. The fluorescence imaging system of claim 144, wherein first recognition molecule and/or the second recognition molecule emits fluorescent light having spectral content in each of the first optical band and the second optical band.

146. The fluorescence imaging system of any one of claims 138 to 145, further comprising an excitation light source configured to illuminate the sample with excitation light to excite the sample to emit the fluorescent light.

147. A fluorescence imaging device, comprising:
a first fluorescence camera;
a second fluorescence camera; and
optical components configured to receive fluorescent light emitted by a sample and divide the fluorescent light between a first optical path that includes the first fluorescence camera and a second optical path that includes the second fluorescence camera.

148. The fluorescence imaging device of claim 147, wherein the optical components are configured to:
divide the fluorescent light into a first portion of the fluorescent light contained within a first optical band and a second portion of the fluorescent light contained within a second optical band;
transmit the first portion of the fluorescent light to the first fluorescence camera along the first optical path; and
transmit the second portion of the fluorescent light to the second fluorescence camera along the second optical path.

149. The fluorescence imaging device of claim 148, wherein the optical components comprise a dichroic mirror optically coupled to the first optical path and the second optical path, the dichroic mirror having a cutoff wavelength between the first optical band and the second optical band.

150. A fluorescence imaging system comprising:
the fluorescence imaging device of claim 148 or 149; and
processing circuitry configured to determine fluorescence information of the sample, the fluorescence information comprising and/or based on a relationship between intensity of the first portion of the fluorescent light and intensity of the second portion of the fluorescent light.

151. The fluorescence imaging system of claim 150, wherein the relationship comprises a ratio of the intensity of fluorescent light captured by the first fluorescence camera with respect to the intensity of fluorescent light captured by the second fluorescence camera.

152. The fluorescence imaging system of claim 150, wherein the relationship comprises a proportion of the intensity of the first portion of the fluorescent light and/or a proportion of the intensity of the second portion of the fluorescent light.

153. The fluorescence imaging system of claim 150, wherein the processing circuitry is configured to determine that the relationship associates the intensity of the first portion of the fluorescent light and the intensity of the second portion of the fluorescent light with a constrained vector space.

154. The fluorescence imaging system of any one of claims 150 to 153, wherein the processing circuitry is configured to determine the fluorescence information of the sample based on a first image, generated by the first fluorescence camera, and a second image, generated by the second fluorescence camera.

155. The fluorescence imaging system of any one of claims 150 to 154, wherein the processing circuitry is configured to discriminate between a first recognition molecule and a second recognition molecule in the sample based on the relationship.

156. The fluorescence imaging system of claim 155, wherein the first recognition molecule and/or the second recognition molecule emits fluorescent light having spectral content in each of the first optical band and the second optical band.

157. The fluorescence imaging system of any one of claims 150 to 156, further comprising an excitation light source configured to illuminate the sample with excitation light to excite the sample to emit the fluorescent light.

158. A fluorescence imaging system comprising:
a plurality of sample well regions;
a controller configured to:
select a sample well region of the plurality of sample well regions; and
dispense a sample, reagent and/or buffer into the sample well region; and
a fluorescence imaging device configured to capture fluorescent light emitted from the sample well region following dispensation of the sample, reagent, and/or buffer.

159. The fluorescence imaging system of claim 158, wherein:
the controller is configured to select a first sample well region of the plurality of sample well regions as the sample well region and dispense the sample, reagent, and/or buffer into the first sample well region; and
the controller is further configured to select a second sample well region of the plurality of sample well regions and dispense a sample, reagent, and/or buffer into the second sample well region following dispensation of the sample, reagent, and/or buffer into the first sample well region.

160. The fluorescence imaging system of claim 159, wherein the controller is configured to dispense the sample, reagent, and/or buffer into the second sample well region during at least a portion of the fluorescence imaging device capturing fluorescent light emitted from the first sample well region.

161. The fluorescence imaging system of any one of claims 158 to 160, wherein the controller is configured to dispense the reagent into the sample well region, and the reagent comprises at least one member selected from a group consisting of:
a composition comprising a terminal amino acid recognition molecule;
a composition comprising a catalyst for removal of a terminal amino acid of a polypeptide immobilized to a surface of a sample well in the sample well region; and
a composition comprising a terminal amino acid modifier.

162. The fluorescence imaging system of claim 161, wherein:
the controller is configured to dispense the composition comprising the terminal amino acid modifier into the sample well region when the sample well region contains a catalyst for removal of a terminal amino acid of a polypeptide immobilized to a surface of a sample well in the sample well region; and
the catalyst comprises an enzyme having increased catalytic activity for removal of a terminal amino acid having been modified by the terminal amino acid modifier.

163. The fluorescence imaging system of any one of claims 158 to 162, wherein:
the controller is configured to dispense a composition comprising a terminal amino acid recognition molecule into the sample well region; and
the fluorescence imaging device is configured to capture fluorescent light emitted from the sample well region after the composition comprising the terminal amino acid recognition molecule is dispensed into the sample well region.

164. The fluorescence imaging system of claim 163, wherein the composition comprises a plurality of terminal amino acid recognition molecules configured to, when excited, emit different fluorescent light, respectively.

165. The fluorescence imaging system of claim 164, wherein the different fluorescent light comprises fluorescent light having different spectral content.

166. The fluorescence imaging system of any one of claims 158 to 165, further comprising an excitation light source configured to illuminate the sample well region to excite emission of the fluorescent light.

167. The fluorescence imaging system of any one of claims 158 to 166, wherein the controller is further configured to:
detect an amount of the sample, reagent, and/or buffer in the sample well region; and
in response to determining that the amount is below a predetermined amount, dispense an additional amount of the sample, reagent, and/or buffer, respectively.

168. A fluorescence imaging and/or processing system, comprising:
processing circuitry configured to:
obtain a first image comprising a first plurality of pixel values indicating intensity of fluorescent light received at respective pixels of a fluorescence imaging device; and
transform the first image into a second image comprising a second plurality of pixel values indicating intensity of fluorescent light received from respective sample wells of a sample well member.

169. The fluorescence imaging and/or processing system of claim 168, further comprising the fluorescence imaging device, wherein the processing circuitry is configured to receive the first image from the fluorescence imaging device.

170. The fluorescence imaging and/or processing system of claim 168 or 169, further comprising the sample well member.

171. The fluorescence imaging and/or processing system of claim 170, wherein the sample well member comprises a plurality of sample well regions, and the second plurality of pixel values indicate intensity of fluorescent light received from respective sample wells of a sample well region of the plurality of sample well regions.

172. The fluorescence imaging and/or processing system of any one of claims 168 to 171, wherein the processing circuitry is configured to transform the first image into the second image at least in part by distributing intensity indicated in the first plurality of pixel values among the second plurality of pixel values.

173. The fluorescence imaging and/or processing system of claim 172, wherein the processing circuitry is configured to distribute the intensity indicated in the first plurality of pixel values among the second plurality of pixel values based on a determined relationship between light received by the respective pixels of the fluorescence imaging device and light emitted by the respective sample wells.

174. The fluorescence imaging and/or processing system of claim 173, wherein the processing circuitry is configured to transform the first image into the second image at least in part by distributing intensity indicated in a first pixel value of the first plurality of pixel values into a second pixel value and a third pixel value of the second plurality of pixel values.

175. The fluorescence imaging and/or processing system of claim 173 or 174, wherein the processing circuitry is further configured to distribute the intensity indicated in the first plurality of pixel values among the second plurality of pixel values further based on a determined alignment between the sample well member and the fluorescence imaging device.

176. The fluorescence imaging and/or processing system of any one of claims 168 to 175, wherein the processing circuitry is further configured to determine alignment between the sample well member and the fluorescence imaging device based on an indicated location in the first image of an alignment feature of the sample well member.

177. The fluorescence imaging and/or processing system of any one of claims 168 to 176, wherein the processing circuitry is configured to receive the first image from a first fluorescence camera of the fluorescence imaging device, and the processing circuitry is further configured to:
obtain a third image comprising a third plurality of pixel values indicating intensity of fluorescent light received at respective pixels of a second fluorescence camera of the fluorescence imaging device; and
transform the third image into a fourth image comprising a fourth plurality of pixel values indicating intensity of fluorescent light received from the respective sample wells of the sample well member.

178. The fluorescence imaging and/or processing system of claim 177, wherein the processing circuitry is further configured to combine the second plurality of pixel values with the fourth plurality of pixel values to determine:
a total intensity of fluorescent light emitted by each of the respective sample wells; and/or
a relationship between intensity of the fluorescent light indicated in the second plurality of pixel values and intensity of the fluorescent light indicated in the fourth plurality of pixel values.

179. The fluorescence imaging and/or processing system of claim 178, wherein the relationship comprises a ratio of the intensity of the fluorescent light indicated in the second plurality of pixel values with respect to the intensity of the fluorescent light indicated in the fourth plurality of pixel values, respectively.

180. The fluorescence imaging and/or processing system of claim 178, wherein the relationship comprises a proportion of the intensity of the fluorescent light indicated in the second plurality of pixel values and/or of the intensity of the fluorescent light indicated in the fourth plurality of pixel values.

181. The fluorescence imaging and/or processing system of claim 178, wherein the processing circuitry is configured to determine that the relationship associates the intensity of the fluorescent light indicated in the second plurality of pixel values and the intensity of the fluorescent light indicated in the fourth plurality of pixel values with a constrained vector space.

182. The fluorescence imaging and/or processing system of any one of claims 178 to 181, wherein the processing circuitry is configured to combine the second plurality of pixel values with the fourth plurality of pixel values prior to and/or at least in part while receiving, from the fluorescence imaging device, a fifth fluorescence image comprising a fifth plurality of pixels indicating intensity of fluorescent light received at the respective pixels of the first fluorescence camera.

183. The fluorescence imaging and/or processing system of any one of claims 177 to 182, wherein the fluorescent light indicated in the first plurality of pixel values is contained within a first optical band and the fluorescent light indicated in the second plurality of pixel values is contained within a second optical band.

184. A fluorescence imaging and/or processing system, comprising:
processing circuitry configured to:
obtain a fluorescence image captured by a fluorescence imaging device indicating fluorescent light emitted from a sample well member;
determine, based on the fluorescence image, a position of the fluorescence imaging device with respect to the sample well member; and
output a signal indicating an extent of alignment and/or misalignment between the fluorescence imaging device and the sample well member.

185. The fluorescence imaging and/or processing system of claim 184, further comprising a controller configured to adjust the position of the fluorescence imaging device with respect to the sample well member in response to the signal indicating misalignment between the fluorescence imaging device and the sample well member.

186. The fluorescence imaging and/or processing system of claim 185, wherein the controller is configured to control a motor to adjust the position of the fluorescence imaging device to an extent corresponding to the extent of alignment and/or misalignment between the fluorescence imaging device and the sample well member.

187. The fluorescence imaging and/or processing system of any one of claims 184 to 186, further comprising the fluorescence imaging device, wherein the processing circuitry is configured to receive the fluorescence image from the fluorescence imaging device.

188. The fluorescence imaging and/or processing system of any one of claims 184 to 187, wherein the processing circuitry is configured to determine the position of the fluorescence imaging device with respect to the sample well member based on an indicated location in the fluorescence image of an alignment feature of the sample well member.

189. The fluorescence imaging and/or processing system of any one of claims 184 to 188, wherein the fluorescence image comprises a plurality of pixel values indicating intensity of fluorescent light received at respective pixels of the fluorescence imaging device.

190. The fluorescence imaging and/or processing system of claim 189, wherein the processing circuitry is further configured to determine a target position of the fluorescence imaging device with respect to the sample well member, at which intensity indicated in the plurality of pixel values is substantially maximized and/or at which intensity from each of a plurality of sample wells of the sample well member is indicated in the plurality of pixel values.
